(19) United States
(12) Patent Application Publication
(10) Pub. No.: US 2006/0019915 A1
(43) Pub. Date: Jan. 26, 2006

(54) Method to modulate the immune system with a novel guanine nucleotide exchange factor

(76) Inventors: Jessica C. Fanzo, New York, NY (US); So Young Jang, New York, NY (US); Sanjay Gupta, Bergenfield, NJ (US); Ayesha Siddiq, Elmsford, NY (US); Steven Greenburg, Englewood, NJ (US); Alessandra B. Pernis, Mountain Lake, NJ (US)

Correspondence Address: BROWN RAYSMAN MILLSTEIN FELDER & STEINER LLP, 900 THIRD AVENUE, NEW YORK, NY, 10022, US

(21) Appl. No.: 11/066,012

(22) Filed: Feb. 24, 2005

Related U.S. Application Data
(60) Provisional application No. 60/548,144, filed on Feb. 25, 2004.

(51) Int. Cl. A61K 48/00; A61K 38/54

(52) U.S. Cl. 514/44; 514/2

(57) ABSTRACT

The present invention provides a method of modulating T cell receptor (“TCR”) dependant regulation of a signaling factor in a T cell, and a method of modulating the proliferation and/or differentiation of a T cell, which includes administering to the T cell an IBP modulator in an amount effective to modulate the function of IBP. The present invention further provides a method and a kit for identifying a modulator of IBP-Lck interaction, a modulator of IBP-PI(3,4,5)P₃ interaction, a modulator of a signaling factor in a T cell. Also provided are compositions containing an IBP modulator.

### CROSS-REFERENCE TO RELATED APPLICATIONS

This application claims the benefit of U.S. Provisional Application No. 60/548,144, filed on Feb. 25, 2004, which is incorporated herein by reference.

### STATEMENT OF GOVERNMENT INTEREST

This invention was made with government support under NIH Grant Nos. R01 HL-62215 and PO1 AI50514-01. As such, the United States government has certain rights in this invention.

### BACKGROUND OF THE INVENTION

Engagement of the T cell receptor (TCR) initiates a complex cascade of biochemical events that culminates in the expansion and differentiation of T cells (1, 2). Activation of protein tyrosine kinases (PTKs) of the Src family, Lck and Fyn, constitutes one of the most proximal and crucial signaling events that couples receptor engagement to downstream biochemical pathways (46). These Src family kinases phosphorylate specific tyrosine residues within the immunoreceptor tyrosine-based activation motifs of the CD3 and TCR □ chain leading to the recruitment and activation of additional kinases, adaptor proteins, and enzymes. Pharmacological and genetic studies have indicated that stimulation of the activity of one enzyme, phosphoinositide 3-kinase (PI3K), is particularly important in mediating the propagation and amplification of the TCR-mediated signaling cascade (47, 48). The products of PI3K, PI(3,4,5)P₃, and PI(3,4)P₂ bind to pleckstrin homology (PH) domains contained in a variety of crucial signaling intermediates inducing the relocalization of these proteins to specific areas of the plasma membrane.

The precise biochemical events triggered by engagement of the T cell receptor differ depending on the strength of the signal received by the T cell (46). For instance, optimal activation of ERK1/2 is usually detected in response to strong but not weak agonists. These differences in TCR-mediated signals can ultimately lead to the acquisition of distinct T cell effector functions. Thus high potency signals have been associated with the generation of THI cells (which produce 1L-2 and IFN-γ) while low potency signals have been linked to the generation of TH2 cells (which produce 1L-4, IL-5, IL-10 and 1L-13). Although the mechanisms utilized by cytokines to direct TH differentiation have been extensively investigated (47, 48), the molecular machinery that connects TCR engagement to distinct effector pathways is less well understood.

T cell recognition of antigen presenting cells (APCs) results in the formation of a specialized interface, termed the immunological synapse (IS) (9, 10, 11). The immunological synapse may function to integrate and/or stabilize TCR-generated signaling pathways and to promote the restricted delivery of secretory products, such as cytokines, to the target cell. Assembly of the immunological synapse is accompanied by the large scale redistribution of receptors and signaling proteins, processes that are critically dependent on TCR-mediated actin cytoskeletal remodeling and polarization. Actin cytoskeletal reorganization in the immunological synapse requires the activity of the Rho family of GTPases (9, 10).

The complex biochemical events triggered by TCR engagement are closely linked to the reorganization of the actin cytoskeleton (9, 10, 11). TCR-mediated cytoskeletal remodeling is necessary for the proper assembly of the immunological synapse (IS) (49, 50, 14), which is crucial for the optimal delivery of TCR-induced signals. Weakly binding ligands are much less efficient in inducing formation of the IS than stronger ligands (15, 24). Actin cytoskeletal remodeling requires the activity of the Rho family of GTPases, which includes Rac and Cdc42 (51, 21, 22) and these GTPases are essential for the appropriate development and function of T cells (23, 52). The ability of Rho GTPases to control T cell function extends beyond their role as key regulators of cytoskeletal reorganization. In particular, Rho GTPases regulate gene expression via their ability to activate mitogen activated protein (MAP) kinases (53). A major class of proteins responsible for the activation of Rho GTPases is the Dbl family of guanine nucleotide exchange factors (GEFs) (27). One member of this family, Vav, plays a key role in T cell cytoskeletal reorganization and its deficiency leads to profound developmental and functional defects in T cells (26, 29, 54). Although it has been proposed that cells utilize different GEFs to tightly control Rho GTPase-.mediated responses (55, 56), no GEFs other than Vav have been implicated in T cell activation.

The inventors have cloned a protein termed IBP (IRF-4 Binding Protein), which exhibits significant homology with SWAP-70 (57), a novel type of Rac-GEF (58). SWAP-70 expression is confined predominantly to mature B lymphocytes and SWAP-70 deficient mice exhibit detects in IgE production (59) suggesting that SWAP-70 plays a unique role in specific B cell activation pathways. In contrast to SWAP-70, which is absent in T cells (60), IBP is highly expressed in this cellular compartment (57). The inventors have also demonstrated that lack of IBP leads to defects in the production of IFN-γ and IL-2 but not IL-4. IBP deficient T cells respond suboptimally to TCR engagement as evidenced by impairments in ERK1/2 activation. The inventors have further shown that IBP mutant T cells display defective cytoskeletal reorganization and synapse formation. These defects can be rescued by a wild-type IBP protein but not by an IBP mutant lacking GEF activity. Thus the inventors have shown that IBP is a novel type of GEF required for the acquisition of the full effector potential of T cells.

### SUMMARY OF THE INVENTION

The present invention provides a method of modulating T cell receptor (TCR) dependant regulation of an effecting factor in a T cell, which includes administering to the T cell an IBP modulator in an amount effective to modulate the function of IBP. The effecting factor may be selected from the group consisting of CD25, CD69, Cdc42, ERK1, ERK2, actin, c-Fos, IFN-γ, IgE, IgG, IL-2, LAT, Rac1, and ZAP-70.

The present invention further provides a method of modulating the proliferation and/or differentiation of a T cell (e.g., a TCR dependent T cell differentiation), which includes administering to the T cell an IBP modulator in an amount effective to modulate the function of IBP. In one embodiment, the TCR dependent T cell differentiation is a TH1 differentiation, which may be inhibited by down-regulating IBP in the T cell using an IBP modulator. In another embodiment, the TCR dependent T cell differentiation is a TH2 differentiation, which may be enhanced by up-regulating IBP in the T cell using an IBP modulator.

Additionally, the present invention provides a method for identifying a modulator of IBP-Lck interaction, which includes (a) administering a candidate agent and IBP to an in vitro system containing Lck; and (b) determining the effect of the candidate agent on Lck catalyzed IBP phosphorylation. Also provided is a kit for use in identifying a modulator of IBP-Lck interaction, which includes (a) IBP; (b) Lck; (c) at least one kinase assay reagent; and (d) instructions for using the kit.

In addition, the present invention provides a method for identifying a modulator of IBP-PI(3,4,5)P₃ interaction, which includes (a) administering a candidate agent and IBP to an in vitro system containing PI(3,4,5)P₃; and (b) determining the effect of the candidate agent on IBP-PI(3,4,5)P₃ interaction. Also provided is a kit for use in identifying a modulator of IBP-PI(3,4,5)P₃ interaction, which contains (a) IBP; (b) PI(3,4,5)P₃; and (c) instructions for using the kit.

Moreover, the present invention provides a method for identifying a modulator of an effecting factor in a T cell, and the modulator identified therewith, which includes (a) administering a candidate agent to the T cell having the effecting factor, where the candidate agent may be selected from the group consisting of an expression vector having a nucleic acid encoding a candidate IBP modulating agent, a candidate IBP gene expression silencing agent, a candidate IBP gene expression enhancing agent, a candidate IBP inhibitor, and a candidate IBP augmenter; and (b) determining the effect of the candidate agent on the effecting factor. The effecting factor may be at least one of CD25, CD69, Cdc42, ERK1, ERK2, F-actin, c-Fos, IFN-γ, IgE, IgG, IL-2, LAT, Rac1, and ZAP-70.

The present invention also provides a composition which contains an IBP modulator, where the IBP modulator modulates TCR dependant regulation of at least of an effecting factor in a T cell, and where the effecting factor may be selected from the group consisting of CD25, CD69, Cdc42, ERK1, ERK2, F-actin, c-Fos, IFN-γ, IgE, IgG, IL-2, LAT, Rac1, and ZAP-70.

The present invention further provides a composition which contains an IBP modulator, wherein the IBP modulator modulates the proliferation and/or differentiation of a T cell.

The present invention further provides a composition which contains an IBP modulator, wherein the IBP modulator modulates the proliferation and/or differentiation of a T cell.

Additional aspects of the present invention will be apparent in view of the description which follows.

### BRIEF DESCRIPTION OF THE FIGURES

FIG. 1 depicts the differentiation of T lymphocyte in IBP^{-/-} mice. (A) IBP protein expression in IBP^{+/+}, IBP^{+/-}, and IBP^{-/-} splenocytes and thymocytes. Total cell lysates (20 μg) were prepared from splenocytes and thymocytes and were probed with an anti-IBP antibody reactive against the C-terminus of IBP (upper panel). Extracts from NIH 3T3 and EL4 cells were used as negative and positive controls respectively. Reprobing with a □-actin antibody is shown as a loading control (lower panel). (B) Flow cytometric analysis of lymphocytes from 6-week-old wild-type and IBP^{-/-} mice. Single cell suspensions from thymus (upper panel), spleen (middle panel), and lymph nodes (lower panel) were stained with antibodies against CD4 and CD8. Percentages of positive cells within each quadrant are shown. Results are representative of six different experiments. (C) CD3 expression in IBP^{-/-} T cells. Single cell suspensions from thymus (upper panel), spleen (middle panel), and lymph nodes (lower panel) from 6-week-old wild-type and IBP mutant mice were stained with antibodies against CD4 and CD3. Cell staining was analyzed by flow cytometry. Histograms represent gated CD4⁺ populations. (D) Flow cytometric analysis of T lymphocytes in IBP^{-/-} mice. Single cell suspensions from spleen and lymph nodes of 6-week-old wild-type and IBP^{-/-} mice were stained with antibodies against CD4 and CD44 (upper panel) or CD4 and CD62L (lower panel). Cell staining was analyzed by flow cytometry. Histograms represent gated CD4+ populations.

FIG. 2 illustrates that IBP is a critical regulator of T cell effector function. A) Proliferation of T cells from wt and IBP mutant mice. Cells were stimulated with plate-bound anti-CD3ε (2C1I) (I mg ml) and soluble anti-CD28 (1 μg/ml) antibodies, or with PMA (SO ng/mt) plus ionomycin (1 μM) for 48 h. The culture was then pulsed with j3Illthymidine for 18 hours. B) IL-2 production by wt and IBP mutant T cells. Purified CD4+ T cells were stimulated with immobilized anti-CD3e antibody (1 μg/ml) alone or together with soluble anti-CD28 antibody (1 mg/ml) (left panel), or with PMA (50 ng/ml) and ionomycin (1 μM) (right panel) for 24 hours. IL-2 levels in culture supernatants were determined by ELISA. C) IFN-γ and 1L-4 production by wt and IBP mutant T cells. Cells were stimulated as in (C) for 48 hours. Production of IFN-γ (right panel) and IL-4 (left panel) was measured by ELISA.

FIG. 3 shows that TH differentiation and serum Ig levels in IBP+/+ mice. A) In vitro differentiation of IBP+/+ IB1−/− name TH cells. Naive CD4 T cells were isolated from wild-type and IBP-1-mice and differentiated in vitro under unskewed (U), TH1, or TH2 conditions. After 7 days of culture, U, TH1 and TH2 cells from IBP+/+ and IBP^{-/-} mice were stimulated with anti-CD3 antibody for 24 hours and supernatants analyzed for cytokine production. IFN-γ (left panel) and IL-4 (right panel) production was measured by ELISA. All experiments are representative of at least five independent experiments, B) Expression levels of T-bet and GATA-3 in IBP−/− T cells. Western blot analysis showing the expression of T-bet (upper panel) and GATA-3 (middle panel) in purified nave CD4⁺ T cells from IBP^{+/+} and IBP^{-/-} mice cultured for 7 days under unskewed (U), THI-, and TH2-polarizing conditions. Extracts from a TH1 clone (AE7) and a TH2 clone (D10) were utilized as controls for T-bet and GATA-3 expression, respectively. Reprobing with a B-actin antibody (lower panel) is shown as a loading control. C) Basal immunoglobulin levels in IBP′/′ mice. Serum immunoglobulin levels from non-immunized 6-week-old IBP^{-/-} (filled circles) and IBP^{-/-} (open circles) mice were determined by isotype-specific ELISA. Each symbol represents one mouse. Horizontal bars are drawn through the median value of each group. *, p<0.005 (IBP^{-/-} vs wild-type). D) 1BP^{+/+} (tilled . circles) and IBP^{-/-} (open circles) mice were immunized with NP-KLH. Relative amounts of NP-specific IgG₁, IgG₂ₐ, IgG₃, antibodies and total IgE levels at the indicated times after primary immunization were determined by FL.ISA. Each symbol represents one mouse Horizontal bars are drawn through the median value of each group. *, p<0.05; **, p<0.005 (IBP^{-/-} wild-type).

FIG. 4 demonstrates that initial TCR signaling is normal in IBP^{-/-} T cells. A) Lck and ZAP-70 activation in IBP^{-/-} T cells. Activation of Lck and ZAP-70 was detected by Western blotting utilizing antibodies specific for Tyr 394 phosphorylated Lck (upper panel), and Tyr 319 phosphorylated ZAP-70 (middle panel). Reprobing with an antibody against total Lck is shown as a loading control (lower panel). B) TCR-mediated calcium mobilization in IBP+/+ and IBP^{-/-} T cells. Lymph node cells were loaded with Fura-red and Fluo-4 and surface stained with APC-labeled anti-CD4 Ab. Cells were then pre-coated with 51 g anti-CD3ε (2C11) antibody and cross-linked with goat anti-hamster Ig. Histogram data are presented as a median ratio of calcium mobilization gated on CD4+ cells as measured by flow cytometry. The black line represents IBP^{+/+} T cells and the gray line represents 1B^{-/-} T cells. Arrowhead indicates the addition of cross-linking antibody.

FIG. 5 sets forth the activation of ERK1/2 is impaired in IBP^{-/-} T cells. A) ERK activation in IBP^{+/+} and IBP^{-/-} T cells. Cells were stimulated with anti-CD32 Ab for the indicated times, or PMA (50 ng/ml) for 2 min as a control. Whole cell lysates were prepared in 1% NP-40 and levels of active ERK1/ERK2 were detected by Western blotting using an antiphospho-ERK antibody (upper panel). Total ERK1/ERK2 levels are shown in the lower panel. B) Induction of c-Fos in IBP-′T cells. Primed T cells from IBP^{+/+} and IBP^{-/-} mice were stimulated with anti-CD3ε antibody (5 mg/ml) and anti-CD28 antibody (5 mg/ml) for 0, 1 or 2 h. Whole cell lysates were prepared in 1% NP-40 and levels of c-Fos were detected by Western blotting using an anti-c-Fos antibody (upper panel). Re-probing with a β-actin antibody is shown as a loading control (lower panel).

FIG. 6 shows that IBP^{-/-} T cells display impaired actin polymerization and synapse formation. A) Actin polymerization in IBP^{-/-} CD4+ T cells. CD4+ T cells were stimulated with anti-CD3ε Ab, stained with FITC-labeled Phalloidin and F-actin levels measured by flow cytometry. Results are representative of four different experiments. B) Formation of the immunological synapse in IBP^{-/-} CD4⁺ T cells. CD4⁺ T cells were conjugated with A20 mouse B lymphoma cells loaded with or without 2 mg/ml SEE. After 2, 5 and 10 min of T and B cell contact at 37° C., cells were fixed, permeabilized, and stained with antibodies against Tyr493 phosphorylated ZAP-70 (red) and CD4 (green). Conjugates were than examined using laser scanning confocal microscopy. Panel I and II show representative images for conjugates (Phase), phospho-ZAP-70 (red) and CD4 (green) at 0.5 min in IBP+/+ and IBP^{-/-} T cells, respectively. C) T cells engaged in conjugates and displaying accumulation of either phosphorylated ZAP-70 or phosphorylated LAT were scored under a fluorescence microscope. Approximately 150 conjugates were counted per time point in at least three independent experiments. T and B cell conjugates without SEE (upper panels) and with SEE (lower panels) are shown.

FIG. 7 demonstrates that wild-type IBP, but not a mutant lacking the DH domain, rescues the defects in cytoskeletal rearrangement and IFN-γ production of IBP^{-/-} T cells. A) Actin polymerization in IBP^{-/-} CD4⁺ T cells reconstituted with wild-type or mutant IBP retroviral vectors. Naive CD4⁺ T cells were infected with control YFP-RV (empty vector), wild-type IBP-expressing (IBP-RV) or IBP-Δ313-631-expressing retrovirus (IBP-Δ313-631-RV). Cells were harvested after 5 days and restimulated with anti-CD3ε Ab followed by cross-linking with goat anti-hamster Ig at 4° C. (control) or 37° C. Cells were then stained with biotin-labeled Phalloidin followed by streptavidin-PE and actin polymerization was measured by flow cytometry. B) Intracellular cytokine staining in IBP-deficient T cells reconstituted with wt or mutant IBP retroviral vectors. Cells were infected and stimulated as described in (A). Five days after primary stimulation, cells were restimulated with 1 mg/ml anti-CD3ε Ab for 4 h. Cells were then harvested, fixed, permeabilized, and stained with APC-conjugated anti-IFN-γ antibody. Data are shown as dot plot analysis of YFP (horizontal axis) and IFN-γ (vertical axis) expression. The % values shown represent the percentages of YFP⁺ cells that are IFN-γ positive.

FIG. 8 shows the hematopoietic cellularity in IBP^{-/-} mice. Mean values±one standard deviation are shown. Total thymocytes, splenocytes, lymph node cells (mesenteric, axillary and inguinal), and bone marrow cells (two femurs) from IBP +/+ and −/− mice were isolated from six-week-old mice and counted. Percentage of cells stained with antibodies to CD4, CD8, CD3, and B220 were determined by flow cytometry. Wild type mice were littermate and sex-matched.

FIG. 9 shows IBP is tyrosine phosphorylated in response to TCR engagement in an Lck-dependent manner. A, schematic of domains of human IBP and SWAP-70 proteins. The location of the potential Lck-mediated tyrosine phosphorylation site in IBP is indicated. B, Jurkat T cells (J-Tag) were either left unstimulated or stimulated with cross-linked anti-CD3 mAb (OKT3) for the indicated time periods. Whole cell lysates were then prepared and immunoprecipitated (IP) with an anti-IBP antiserum. The immunoprecipitates were resolved by 7% SDS-PAGE and then analyzed by Western blotting using an anti-phosphotyrosine antibody (4G10) (upper panel). The blot was later stripped and reprobed with the anti-IBP antiserum to ensure equal loading of immunoprecipitates (lower panel). C, purified wild-type (wt) HA-tagged IBP (150 ng) was subjected to in vitro kinase reactions either alone (IBP) or together with purified Lck kinase (5 units) in the presence of [γ-³²P]ATP. Control kinase reactions included [γ-³²P]ATP alone (ATP) or purified Lck alone (Lck). The reactions were resolved by 7% SDS-PAGE. The phosphorylated products were subsequently detected by autoradiography. D, 293T cells were transiently transfected with an empty vector (CONTROL) or with an expression vector for either wild-type (wt) IBP or the IBPY210F mutant. Whole cell lysates were prepared and immunoprecipitated with an anti-IBP antiserum. The immunoprecipitates were extensively washed and then subjected to in vitro kinase reactions with purified Lck kinase (5 units) in the presence of [γ-³²P]ATP. One-half of each reaction was resolved by 7% SDS-PAGE, and the phosphorylated products were subsequently detected by autoradiography (left panel). The remaining half of the reaction was analyzed by Western blotting with the anti-IBP antibody to ensure for equivalent expression levels of wild-type and mutant IBP proteins in the different transfectants (right panel). E, cells from wild-type Jurkat (JE6-1 (Lck⁺)) and a Jurkat variant (J. CaM1.6 (Lck⁻)) were stimulated with cross-linked anti-CD3 mAb (OKT3) for the indicated periods of time. Whole cell lysates were obtained, immunoprecipitated with an anti-IBP Ab, and then analyzed by Western blotting as described for A.

FIG. 10 shows interaction of IBP with phosphoinositides upon Lck stimulation. A, 293T cells were transiently cotransfected with appropriate vector controls (CONTROL) or with a wild-type IBP expression vector, together with either an empty vector (wt IBP) or a constitutively active Lck expression vector (wt IBP+LckY505F). Whole cell lysates were prepared and incubated with PI(4,5)P₂, PI(3,4)P₂, or PI(3,4,5)P₃ analogue beads. The bead-bound proteins were resolved by 7% SDS-PAGE and then analyzed by Western blotting using an anti-IBP Ab (upperpanel). The blot was later stripped and reprobed with an anti-PLC.1 Ab as a control (lower panel). B, 293T cells were transiently cotransfected with an expression vector for either wild-type IBP or the IBP R236C mutant, together with either an empty vector or a constitutively active Lck expression vector (LckY505F). Whole cell lysates were prepared and incubated with PI(4,5)P₂, PI(3,4)P₂, or PI(3,4,5)P₃ analogue beads and then the bound proteins were analyzed by Western blotting using an anti-IBP Ab. C, 293T cells were transiently cotransfected with an expression vector for either wild-type IBP or the IBPY210F mutant, together with either an empty vector or a constitutively active Lck expression vector (LckY505F). Whole cell lysates were prepared and incubated with PI(3,4,5)P₃ analogue beads and then the bound proteins were analyzed by Western blotting using an anti-IBP Ab. D, 293T cells were transiently transfected with an empty vector (CONTROL), a wild-type IBP expression vector (wt IBP), or an expression vector encoding an IBP deletion mutant lacking the N-terminal region (IBP ΔN). Whole cell lysates were obtained and incubated with PI(4,5)P₂, PI(3,4)P₂, or PI(3,4,5)P₃ analogue beads and then the bound proteins were analyzed by Western blotting as described above.

FIG. 11 shows recruitment of IBP to the immunological synapse. A, 1×10⁵ Jurkat E6-1 (Lck⁺) or 1×10⁵ J.CaM1.6 (Lck⁻) cells were mixed in a 1:1 ratio with the B cell lymphoma cell line, Raji cells, that had been prepulsed with medium alone or with 5 μg/ml of SEE superantigen. Raji cells were labeled with the 7-methyl-4-chloromethylcoumarin cell tracker dye (Molecular Probes). Cell conjugates were pelleted, plated on poly-L-lysine-coated slides, fixed, permeabilized, and stained with antibodies to IBP and PKC-θ, followed by a secondary staining with Alexa-Fluor 568-conjugated anti-rabbit Ig (red) and FITC-conjugated anti-mouse Ig (green), respectively. Conjugates were then examined by confocal microscopy. Fluorescence and phase images are shown. In the wortmannin-treated samples, T cells had been pretreated with 100 nM wortmannin prior to incubation with APCs. B, quantitative analysis of data presented in A. Cells were scored visually for localization of IBP (solid bars) or PKC-0 (hatched bar) at the synapse. The graphs represent the mean percentages of imaged cells showing IBP or PKC-θ at the synapse±S.D. of two independent scores from three different experiments. Approximately 100 conjugates per stain were examined, and those showing a distinct band of labeling at the contact site were scored.

FIG. 12 shows that IBP colocalizes with the TCR/CD3 cap in primary murine TCR transgenic T cells. Purified T lymphocytes from the D0.11.10 TCR transgenic mouse were incubated with 5 μg/ml anti-CD3ε (145-2C11; Pharmingen) and then an FITC-labeled cross-linking secondary Ab at 4 or 37° C. Cells were then fixed and stained with an antibody against IBP followed by an Alexa-Fluor 568-conjugated secondary antibody staining. Cells were visualized by confocal microscopy. A representative single cell enlarged image before (4° C.) and after (37° C.) activation for 5 min is shown.

FIG. 13 depicts the In vivo activation of Rac1 and Cdc42 by wild-type or mutant IBP proteins. A, a schematic diagram of the IBP deletion mutants utilized in the Rac1/Cdc42 activation assays. B, levels of active Rac1 in cells expressing wild-type or mutant IBP proteins. COS-7 cells were transiently transfected with an empty vector (CONTROL) or with an expression construct for either wild-type IBP or the indicated IBP deletion mutants. Whole cell lysates were prepared and incubated with immobilized GST-PAK1 PBD fusion protein to affinity precipitate activated (i.e. GTP-bound) Rac1. The amount of Rac1-GTP that bound to the GST-PAK1 PBD was visualized by separation on an SDS-polyacrylamide gel followed by Western blotting with an anti-Rac1 antibody. The corresponding total cell lysates used for pull-down assays (INPUT) were simultaneously immunoblotted to ensure for equivalent expression levels of Rac1 in the different transfectants. C, levels of active Cdc42 in cells expressing wild-type or mutant IBP proteins. COS-7 cells were transiently transfected with an empty vector (CONTROL) or with an expression construct for either wild-type IBP or the indicated IBP deletion mutants. Whole cell lysates were prepared and incubated with immobilized GST-PAK1 PBD fusion protein to affinity precipitate activated (i.e. GTP-bound) Cdc42. The amount of Cdc42-GTP that bound to the GST-PAK1 PBD was visualized by separation on an SDS-polyacrylamide gel followed by Western blotting with an anti-Cdc42 antibody. Corresponding total cell lysates used for pull-down assays (INPUT) were simultaneously immunoblotted to ensure for equivalent expression levels of Cdc42 in the different transfectants.

FIG. 14 demonstrates that IBP exhibits GEF activity toward Rac1 and Cdc42 in vitro, which can be modulated by Lck and PI(3,4,5)P₃. A-C, stimulation of GDP dissociation from Rho GTPases by wild-type or mutant IBP proteins. [³H]GDP-loaded Rac1 (A), Cdc42 (B), or RhoA (C) was incubated with GST alone or with either wild-type or mutant GST-IBP fusion proteins, as indicated. The exchange reactions were stopped at the indicated time points, and the amount of [³H]GDP remaining bound to the GTPases was then measured by filtering the quenched reaction samples over nitrocellulose membranes followed by scintillation counting of the membrane-bound radioactivity. The percentage of [³H]GDP remaining bound at each time point was evaluated relative to the amount of radioactivity remaining at time 0 of the respective reaction, which was set to 100% in each experiment. The results shown are representative of at least three independent assays. D, regulation of IBP GEF activity by Lck and PI(3,4,5)P₃. [³H]GDP-loaded Rac1 was incubated with Lck-phosphorylated wild-type HA-IBP (IBP+Lck) in the presence or absence of PI(3,4,5)P₃ (1 μM) for 0 or 40 min. Reactions without IBP (Lck and Lck⁺ PI(3,4,5)P₃) were simultaneously carried out as control. The exchange reactions were then stopped, and the amount of [³H]GDP remaining bound to Rac1 was measured by filter-binding method as described for A-C. The percentage of [³H]GDP remaining bound after incubation for 40 min was evaluated relative to the amount of radioactivity remaining at time 0 of the respective reaction, which was set to 100% in each experiment. The results shown are representative of at least three independent assays.

### DETAILED DESCRIPTION OF THE INVENTION

As used herein and in the appended claims, the singular forms “a,” “an,” and “the” include plural references unless the content clearly dictates otherwise. Thus, for example, reference to “an effecting factor” includes a plurality of such factors and equivalents thereof known to those skilled in the art, and reference to “the modulator” is a reference to one or more modulators and equivalents thereof known to those skilled in the art, and so forth. All publications, patent applications, patents, and other references mentioned herein are incorporated by reference in their entirety.

The present invention provides a method of modulating T cell receptor (“TCR”) dependant regulation of an effecting factor in a T cell, which includes administering to the T cell an IBP modulator in an amount effective to modulate the function of IBP, where the effecting factor may be CD25, CD69, Cdc42, ERK1, ERK2, actin, c-Fos, IFN-γ, IgE, IgG, IL-2, LAT, Rac1, or ZAP-70. As used herein and in the appended claims, the term “regulation” includes any type of regulations, such as, without limitation, regulation of gene expression, regulation of activity, regulation of secretion, regulation of sub-cellular localization, regulation of phosphorylation status, regulation of half-life, regulation of activity, and regulation of assembly.

The IBP modulator may be an IBP inhibiting agent or an IBP enhancing agent. As used herein and in the appended claims, the term “agent” shall include any protein, polypeptide, peptide, nucleic acid (including DNA, RNA, and genes), antibody and fragments thereof, molecule, compound, and any combinations thereof. The function of IBP may be inhibited or enhanced at a number of levels, including, without limitation, the gene level (e.g., by knocking out the IBP gene or introducing into the genome a nucleic acid encoding a gain-of-function IBP or Lck mutant), the gene expression level (e.g., the transcription level, the translation level, or the post-translation level), and the protein level (e.g., by using IBP antagonist, IBP agonist, or IBP antibody). In one embodiment, the IBP modulator may be selected from the group consisting of an expression vector comprising a nucleic acid encoding an IBP modulating agent, a gene knockout vector, a gene expression silencing agent, a gene expression enhancing agent, an IBP inhibitor, and an IBP augmenter. In another embodiment, the IBP modulating agent may be an antisense nucleic acid, an interference RNA, an IBP antibody, a gain-of-function IBP mutant, or a loss-of-function IBP mutant. In yet another embodiment, the gene expression silencing agent may be an antisense nucleic acid and an interference RNA, which reduces the expression of IBP in a T cell. In still another embodiment, an IBP inhibitor may be a suitable small molecule IBP inhibitor, a protein phosphatase, an IBP antibody, and a loss-of-function IBP mutant. In addition, an IBP augmenter may be a suitable small molecule IBP augmenter, a protein kinase, and a gain-of-function IBP mutant. In one embodiment, the IBP augmenter may be Lck. In one embodiment, the function of an IBP may be further enhanced using phosphatidylinositol 3,4,5-triphosphate (“PI(3,4,5)P₃”).

An IBP modulator or modulating agent may be a protein or a polypeptide. Unless otherwise indicated, “protein” or “polypeptide” shall include a protein, protein domain, polypeptide, or peptide, and any fragment, variant, or derivative thereof having polypeptide function. The variants preferably have greater than about 75% homology with the naturally-occurring polypeptide sequence; more preferably, the variants have greater than about 80% homology with the naturally-occurring polypeptide sequence; even more preferably, the variants have greater than about 85% homology with the naturally-occurring polypeptide sequence. Most preferably, the variants have greater than about 90% homology with the naturally-occurring polypeptide sequence. In some embodiments, the homology may be as high as about 95%, 98%, or 99%. These variants may be substitutional, insertional, or deletional variants. The variants may also be chemically-modified derivatives: polypeptides which have been subjected to chemical modification, but which retain the biological characteristics of the naturally-occurring polypeptide. In one embodiment of the present invention, the polypeptide is mutated such that it has a longer half-life in vivo.

As used herein, two polypeptide or nucleic acid sequences are said to be “identical” if the sequence of amino acid residues or nucleotides, respectively, in the two sequences is the same when aligned for optimum correspondence. Optimal alignment of sequences for comparison may be conducted by inspection or by using one of a number of algorithms, such as the homology alignment algorithm of Needleman and Wunsch (Needleman and Wunsch, A general method applicable to the search for similarities in the amino acid sequence of two proteins. J. Mol. Biol. 48: 443-53, 1970), the local homology algorithm of Smith and Waterman (Smith and Waterman, Comparison in biosequences. Adv. Appl. Math. 2: 482-9, 1981), the Pearson and Lipman algorithm (Pearson and Lipman, Improved tools for biological sequence comparison. Proc. Natl. Acad. Sci. USA 85: 2444-8, 1988), or computerized implementations of these algorithms (e.g., BLAST as offered by the National Center for Biotechnology Information). The degree of homology between two sequences is determined by comparing two optimally-aligned sequences, wherein the percentage is calculated by ascertaining the number of positions at which the identical amino acid residue or nucleic acid base occurs in both sequences, to yield the number of matched positions, dividing the number of matched positions by the total number of positions in the sequence, and multiplying the result by 100 to yield the percentage of sequence identity.

An IBP modulator or modulating agent may be a nucleic acid or polynucleotide. As used herein, a “nucleic acid” or “polynucleotide” includes a nucleic acid, an oligonucleotide, a nucleotide, or a polynucleotide, and any fragment or variant thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. The nucleic acid or polynucleotide may be combined with a carbohydrate, a lipid, a protein, or other materials.

The “complement” of a nucleic acid refers, herein, to a nucleic acid molecule which is completely complementary to another nucleic acid, or which will hybridize to the other nucleic acid under conditions of high stringency. High-stringency conditions are known in the art (see, e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001). Stringent conditions are sequence-dependent, and may vary depending upon the circumstances. As used herein, the term “cDNA” refers to an isolated DNA polynucleotide or nucleic acid molecule, or any fragment, derivative, or complement thereof. It may be double-stranded, single-stranded, or triple-stranded, it may have originated recombinantly or synthetically, and it may represent coding and/or noncoding 5′ and/or 3′ sequences.

As used herein, the term “IBP modulator,” “IBP inhibiting agent,” or “IBP augmenting/enhancing agent,” shall include not only such modulator or agent which may directly modulate the function of IBP, but also modulators or agents which may modulate the function of an up-stream regulator or a down-stream effector of IBP. In one embodiment, TCR dependant up-regulation of an effecting factor in a T cell, such as, CD25, CD69, Cdc42, ERK1, ERK2, F-actin, c-Fos, IFN-γ, IL-2, LAT, Rac1, and/or ZAP-70, may be reduced by administering of an IBP inhibiting agent to the T cell or enhanced by administering of the IBP augmenting agent. In another embodiment, TCR dependant up-regulation of IgE and IgG may be increased by administering of an IBP inhibiting agent or reduced by administering of an IBP augmenting agent.

The present invention further provides a method of modulating the proliferation and/or differentiation of a T cell, which includes administering to the T cell an IBP modulator in an amount effective to modulate the function of IBP. In one embodiment, the proliferation and/or differentiation of a T cell is TCR dependent. In another embodiment, the TCR dependent T cell differentiation is a TH1 differentiation, and where the TH1 differentiation may be inhibited by down-regulating IBP in the T cell using an IBP modulator. In yet another embodiment, the TCR dependent T cell differentiation is a TH2 differentiation, and where the TH2 differentiation may be enhanced by up-regulating IBP in the T cell using an IBP modulator.

Additionally, the present invention provides a method for identifying a modulator of IBP-Lck interaction, which includes (a) administering a candidate agent and IBP to an in vitro system which contains Lck; and (b) determining the effect of the candidate agent on Lck catalyzed IBP phosphorylation. For example, the in vitro system may be constructed using any suitable kinase assay kit known in the art, such as, without limitation, IMAP® Lck Tyrosine Kinase Assay Kit (Molecular Devices Corporation), DELFIA® Tyrosine Kinase kit (PerkinElmer, Inc.), and Tyrosine Kinase Activity Assay Kit (CHEMICON International, Inc.). The Lck catalyzed IBP phosphorylation may be determined using standard molecular biology techniques known in the art, such as, immunoprecipitation, Western blot, ELISA, as well as using commercially available detection kit, such as, Phosphotyrosine Detection Kit (EMD Biosciences, Inc.). The observed changes in Lck catalyzed IBP phosphorylation denote the effect of a candidate agent on the Lck-IBP interaction, e.g., agonistic effect or antagonistic effect. The present method may also be used in a high throughput screening context for identifying a modulator of Lck-IBP interaction. In addition, the present invention also provided a kit for use in identifying a modulator of IBP-Lck interaction, which includes (a) IBP (including, e.g., tagged or un-tagged IBP, functional IBP fragment, expression vectors having a nucleic acid encoding IBP or functional fragment thereof, or a cell suitable for expressing IBP); (b) Lck (including, e.g., tagged or un-tagged Lck, functional Lck fragment, expression vectors having a nucleic acid encoding Lck or functional fragment thereof, or a cell suitable for expressing Lck); (c) at least one kinase assay reagent (e.g., buffer and/or antibody); and (d) instructions for using the kit.

Furthermore, the present invention provides a method for identifying a modulator of IBP-PI(3,4,5)P₃ interaction, which includes (a) administering a candidate agent and IBP to an in vitro system containing PI(3,4,5)P₃; and (b) determining the effect of the candidate agent on IBP-PI(3,4,5)P₃ interaction. For example, the in vitro system may contain a plurality of beads having PI(3,4,5)P₃ (including, e.g., equivalent analogs thereof). The EBP-PI(3,4,5)P₃ interaction may be determined using standard molecular biology techniques known in the art, such as Western blot or ELISA. The observed changes in IBP-PI(3,4,5)P₃ binding denote the effect of a candidate agent on the IBP-PI(3,4,5)P₃ interaction, e.g., agonistic effect or antagonistic effect. The present method may also be used in a high throughput screening context for identifying a modulator of IBP-PI(3,4,5)P₃ interaction. Additionally, the present invention also provided a kit for use in identifying a modulator of IBP-PI(3,4,5)P₃ interaction, which includes (a) IBP; (b) PI(3,4,5)P₃ (including, e.g., a bead containing the same); and (c) instructions for using the kit.

In one aspect, the present invention provides a method for identifying a modulator of an effecting factor (e.g., CD25, CD69, Cdc42, ERK1, ERK2, F-actin, c-Fos, IFN-γ, IgE, IgG, IL-2, LAT, Rac1, and ZAP-70) in a T cell, which includes (a) administering a candidate agent to the T cell which contains the effecting factor; and (b) determining the effect of the candidate agent on the effecting factor, where the candidate agent may be an expression vector containing a nucleic acid encoding a candidate IBP modulating agent, a candidate IBP gene expression silencing agent, a candidate IBP gene expression enhancing agent, a candidate IBP inhibitor, and a candidate IBP augmenter. Also provided is a composition (e.g., a pharmaceutical composition) containing the modulator identified therewith. A pharmaceutical composition may include a pharmaceutically-acceptable carrier. The pharmaceutically-acceptable carrier must be “acceptable” in the sense of being compatible with the other ingredients of the composition, and not deleterious to the recipient thereof. The pharmaceutically-acceptable carrier employed herein is selected from various organic or inorganic materials that are used in pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and/or viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, without limitation, intravenous, intraperitoneal, subcutaneous, intramuscular, and intratumoral (i.e., direct injection into the tumor) injection.

The composition of the present invention may be useful for administering an IBP modulator or an IBP modulating agent to a subject to treat a variety of IBP or T cell related disorders (e.g., infectious disorders and neoplasm). As used herein, the “subject” is an animal, including, without limitation, chicken, bird, cow, dog, human, monkey, mouse, pig, or rat. Also, as discussed above, the term “agent” includes polypeptides (e.g., antibody), nucleic acids, lipids, polysaccharides, and small organic compounds, and derivatives, variants, fragments, and combinations thereof.

The pharmaceutical composition may be provided in an amount effective to treat the disorder in a subject to whom the composition is administered. As used herein, the phrase “effective to treat the disorder” means effective to ameliorate or minimize the clinical impairment or symptoms resulting from the disorder. For example, the clinical impairment or symptoms of the disorder may be ameliorated or minimized by diminishing any pain or discomfort suffered by the subject; by extending the survival of the subject beyond that which would otherwise be expected in the absence of such treatment; by inhibiting or preventing the development or spread of the pathological condition to other tissues or organs; or by limiting, suspending, terminating, or otherwise controlling the proliferation of pathogens or neoplastic cells.

The amount of pharmaceutical composition that may be effective to treat an disorder in a subject will vary depending on the particular factors of each case, including, for example, the type or stage of the pathological condition, the subject's weight, the severity of the subject's condition, and the method of administration. These amounts can be readily determined by a skilled artisan.

In the method of the present invention, the pharmaceutical composition may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, and administration by osmotic pump.

For oral administration, the formulation of the pharmaceutical composition may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the pharmaceutical composition may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above. Where the site of the pathological condition is localized to a particular portion of the body of the subject, it may be desirable to introduce the pharmaceutical composition directly to that area by injection or by some other means (e.g., by intra-tumoral delivery, by local delivery, or by introducing the therapeutic composition into the blood or another body fluid).

For transdermal administration, the pharmaceutical composition may be combined with skin-penetration enhancers, such as propylene glycol, polyethylene glycol, isopropanol, ethanol, oleic acid, N-methylpyrrolidone, and the like, which increase the permeability of the skin to the therapeutic composition, and permit the pharmaceutical composition to penetrate through the skin and into the bloodstream. The pharmaceutical composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The pharmaceutical composition may be administered transdermally, at or near the site on the subject where the neoplasm is localized. Alternatively, the pharmaceutical composition may be administered transdermally at a site other than the affected area, in order to achieve systemic administration.

The pharmaceutical composition of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the pharmaceutical composition.

In accordance with the method of the present invention, the pharmaceutical composition may be administered to a subject who has an IBP or T cell related disorder, either alone or in combination with one or more other therapeutic agents, such as, antibiotics, antiinflammation agents, immunosuppression agents, or antineoplastic drugs. Examples of antibiotics with which the pharmaceutical composition may be combined include, without limitation, penicillin, tetracycline, bacitracin, erythromycin, cephalosporin, streptomycin, vancomycin, D-cycloserine, fosfomycin, cefazolin, cephaloglycin, cephalexin, amphotericin B, gentamicin, tobramycin, and kanamycin, and variants and derivatives thereof. Examples of antineoplastic drugs with which the pharmaceutical composition may be combined include, without limitation, carboplatin, cyclophosphamide, doxorubicin, etoposide, and vincristine. Additionally, when administered to a subject, the pharmaceutical composition may be combined with other therapies, including, without limitation, surgical therapies, radiotherapies, gene therapies, and immunotherapies.

The present invention also provides a composition (e.g., pharmaceutical composition) containing an IBP modulator, where the IBP modulator modulates TCR dependant regulation of an effecting factor (e.g., CD25, CD69, Cdc42, ERK1, ERK2, F-actin, c-Fos, IFN-γ, IgE, IgG, IL-2, LAT, Rac1, and ZAP-70) in a T cell; or where the IBP modulator modulates the proliferation and/or differentiation of a T cell.

By way of example, the IBP modulator or modulating agent of the present invention may be a nucleic acid (e.g., expression vector) which encodes or includes at least one gene-silencing cassette that is capable of silencing the expression of genes that are essential or important for the IBP signaling in a T cell. It is well understood in the art that a gene may be silenced at a number of stages. Examples of gene silencing include, without limitation, pre-transcription silencing, transcription silencing, translation silencing, post-transcription silencing, and post-translation silencing. In one embodiment of the present invention, the gene-silencing cassette encodes or comprises a post-transcription gene-silencing composition, such as antisense RNA or RNAi. Both antisense RNA and RNAi may be produced in vitro, in vivo, ex vivo, or in situ.

For example, the IBP modulating agent of the present invention may be an antisense RNA. Antisense RNA is an RNA molecule with a sequence complementary to a specific RNA transcript, or mRNA, the binding of which prevents further processing of the transcript or translation of the mRNA. Antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein et al., Oligodeoxynucleotides as inhibitors of gene expression: a review. Cancer Res., 48:2659-68, 1998).

Antisense molecules designed to bind to the entire target mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes (see, e.g., U.S. Pat. No. 6,448,080, Antisense modulation of WRN expression; U.S. Patent Application No. 2003/0018993, Methods of gene silencing using inverted repeat sequences; U.S. Patent Application No., 2003/0017549, Methods and compositions for expressing polynucleotides specifically in smooth muscle cells in vivo; Tavian et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J. Biol. Chem., e-publication ahead of print, 2003; Ghosh et al., Role of superoxide dismutase in survival of Leishmania within the macrophage. Biochem. J., 369:447-52, 2003; and Zhang et al., An anti-sense construct of full-length ATM cDNA imposes a radiosensitive phenotype on normal cells. Oncogene, 17:811-8, 1998).

Oligonucleotides antisense to a member of the IBP related signal-transduction pathways/systems may be designed based on the nucleotide sequence of the member of interest. For example, a partial sequence of the nucleotide sequence of interest (generally, 15-20 base pairs), or a variation sequence thereof, may be selected for the design of an antisense oligonucleotide. This portion of the nucleotide sequence may be within the 5′ domain. A nucleotide sequence complementary to the selected partial sequence of the gene of interest, or the selected variation sequence, then may be chemically synthesized using one of a variety of techniques known to those skilled in the art, including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

Once the desired antisense oligonucleotide has been prepared, its ability to prevent or treat IBP related disorders then may be assayed. For example, the antisense oligonucleotide may be administered to a diseased subject, such as a mouse or a human, and its effects on the disease may be determined using standard clinical and/or molecular biology techniques, such as Western-blot analysis and immunostaining.

It is within the confines of the present invention that oligonucleotides antisense to a member of the IBP signal-transduction pathways/systems may be linked to another agent, such as an anti-infection or anti-neoplastic drug. Moreover, antisense oligonucleotides may be prepared using one or more modified bases (e.g., a phosphorothioate), as discussed above, to make the oligonucleotides more stable and better able to withstand degradation.

The IBP modulating agent of the present invention also may be an interfering RNA, or RNAi, including small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi (see, e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAi is produced in vitro, synthetically or recombinantly, and transferred into the microorganism using standard molecular-biology techniques. Methods of making and transferring RNAi are well known in the art (see, e.g., Ashrafi et al., Genome-wide RNAi analysis of Caenorhabditis elegans fat regulatory genes. Nature, 421:268-72, 2003; Cottrell et al., Silence of the strands: RNA interference in eukaryotic pathogens. Trends Microbiol., 11:37-43, 2003; Nikolaev et al., Parc. A cytoplasmic anchor for p53. Cell, 112:29-40, 2003; Wilda et al., Killing of leukemic cells with a BCR/ABL fusion gene RNA interference (RNAi). Oncogene, 21:5716-24, 2002; Escobar et al., RNAi-mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001).

The present invention is described in the following Examples, which are set forth to aid in the understanding of the invention, and should not be construed to limit in any way the scope of the invention as defined in the claims which follow thereafter.

### EXAMPLES

### Example 1

Mice deficient in IBP were generated utilizing a gene trapping strategy (32). Integration of the gene trapping construct occurred in the 1′ intron of the IBP gene resulting in the complete absence of IBP expression. All mice were kept under specific pathogen-free conditions and used between 6 and 12 weeks after birth.

### Example 2

### Flow Cytometry Analysis

Single cell suspensions from thymus, spleen, and lymph nodes were isolated, resuspended in staining buffer (PBS containing 1% BSA, 2 mM EDTA and 0.03%, or CD44 antibodies (Pharmingen) for 30 min on ice. Stained cells were analyzed using FACS Calibur with CELLQuest software (Beckton Dickinson).

### Example 3

### In Vitro T Cell Studies

CD4⁺ T cells were purified from red blood-cell depleted spienocytes by negative selection using the CD4⁺ specific T cell enrichment columns (R & D Systems). The purity of CD4′ T cells was assessed by flow cytometry and was found to be >90%. For proliferation assays. purified CD4⁺ T cells were cultured at 1×10⁵ per well in 96 well plates for 48 hours in culture medium alone or in the presence of either plate-bound anti-CD3Σ Ab (145-2C11) (1 μg/ml) and soluble anti-CD2S mAb (1 μg/ml), or PMA (50 μg/ml) plus ionomycin (1 AM). The cultures were then pulsed with [³H]thymidine (1 uCi/well) for 18 hours and incorporated radioactivity was then measured by scintillation counting. For in vitro TH differentiation experiments, naive CD4⁺ T cells were purified from lymph nodes of wild-type and IBP+/+ mice by negative selection using the CD4⁺ CD62L high specific T cell enrichment columns (R & D Systems). The purity of naive CD4⁺ cells was assessed by flow cytometry and was found to be >90%. Cells were then resuspended at 10⁶ cells/ml in complete medium containing Click's media supplemented with 10% FBS, 2 ruM glutannine, 50 U/ml penicillin, 50 μg/ml streptomycin, and 50 μM B-ME at 37° C. in 5% CO² and stimulated with plate-bound anti-CD3 (1 μg/ml) plus anti-CD28 (2 μg/ml) antibodies. Cells were split 1:4 with fresh medium, plus 200 U/ml 1L-2 on day 3 after primary stimulation and then rested. On day 7, cells were washed, counted, and re-stimulated with I μg/ml anti-CD3 Ab at 10⁶ cells/ml in complete medium. For THI-skewing, 2 ng/ml IL-2 and 10 μg/ml anti-IL-4 Ab were added to the primary culture. For TH2-skewing, 10 ng/ml 1L-4 and 10 μg/ml anti-IFN-γ Ab and 10 μg/ml anti-IL-12 Ab were included. Cell-free supernatants were analyzed for cytokine production by ELISA (Endogen) 24 h after secondary stimulation.

### Example 4

### Immunoprecipitation and Immunoblotting

Lymph node T cells were stimulated with 5 tg of hamster anti-CD3r mAb (145-2011) followed by cross-linking with 10 μg of goat anti-hamster Ig for the indicated times. Whole cell lysates were prepared as previously described (30). To examine Lck and ZAP-70 activation, lymph node T cells were stimulated with 5 ug of biotinylated anti-CD3r mAb (145-2C1 I) and anti-CD4 mAb followed by cross-linking with it) 10 ug of streptavidin for the indicated times. Whole cell lysates were prepared as previously described (30) and activation of Lck and ZAP-70 was analyzed by Western blotting with a phosphospecific Ab against Tyr394 of Lek (33) and Tyr319 of ZAP-70 (Cell Signaling). Activation of ERK1/2 was analyzed by western blotting with a phosphospecific Ab against Thr2021Tyr204 of p44 and p42 ERIK (Cell Signaling). Total levels of c-Fos were analyzed by Western blotting using an antibody against c-Fos (Santa Cruz Biotech).

### Example 5

### Serum Immunoglobulins

Serum IgA, IgM, IgG IgG, IgG₂₆, IgG₃ antibodies and total IgE levels from non-immunized six—week-old mice were determined by isotype-specific ELISA (Southern Biotech and Pharmingen). Mice were immunized with 100 lag of NP-KLI-I (keyhole limpet hemocyanin) alum precipitated by intraperitoneal injection. Blood was collected on day 0, 14 and 28 after immunizations. Relative amounts of NP-specific IgM, IgG, IgG2a, IgG₃ antibodies and total IgE levels at the indicated times after primary immunization were determined by ELISA.

### Example 6

### Immunofluorescence Microscopy

A20 mouse B lymphoma cells were used as the APCs for conjugation with CD4⁺ T cells. A20 cells were pulsed with or without 2 Ag/ml SEE (Toxin Technology, Sarasota, Fla.) for 1 hour at 37° C. To induce conjugate formation, 1×103 B cells were combined with 1×10s purified CD4⁺ T cells (a ratio of 1:1), centrifuged briefly at 500 rpm for 5 min, and incubated at 37° C. for 2 min, 5 min, or 10 min. Conjugates were settled onto poly-L-lysine coated cover slips at RT for 10 min, fixed in 3.7% formaldehyde for 10 min, washed, and pertneabilized with 0.1% Triton X-100/PBS for 10 min at RT. Conjugates were then stained with antibodies to phospho-ZAP-70 (Tyr493 ZAP-70, Cell Signaling), phospho-LAT (Tyr191 LAT, Cell Signaling), or FITC-labeled CD4 followed by a secondary staining with Alexa-Fluor 568-conjugated donkey anti-rabbit Ig (Molecular Probes) for both phospho-ZAP-70 and phospho-LAT. Conjugates were examined by a Zeiss LSM 51′0 laser scanning confocal microscope (Thornwood, N.Y.) with a 100×/13 Plan-Neofluor objective lens. FITC and Alexa-Fluor 568 (in two photon mode) were excited at 488 nm and 543 nm, respectively, and emission was collected at 500-550 nm and above 585 nm, respectively. Clustering of phospho-ZAP-70 and phospho-LAT was analyzed by acquiring Z-series of 10 optical sections with an optical section thickness of approximately 1 micron. A single XY optical section of each conjugate was also acquired. The pixel density of the optical field was fixed at 512×512 pixels. The images were acquired with the maximum signal detection without any significant signal saturation. Image enhancement and analysis were performed using the public domain program NIH Image 1.6 and Adobe Photoshop 6.0. For presentation, the gray-scale of images were linearly scaled (eight-bit). Approximately 150 conjugates were scored visually in the absence or presence of SEE for concentration of phosphorylated ZAP-70, phosphorytated LAT and CO₄ at the cell-cell interface. Polarization was scored positive only if phosphorylated ZAP-70, phosphorylated LAT and CD4 within the T cell was located in close proximity to the B cell membrane. Graphs represent the means±SD from three independent scores of three different experiments.

### Example 7

### Calcium Measurement

Single-cell suspensions of Lymph node cells were loaded with Fura-red and Flue-4 (Molecular Probes, Eugene, Oreg.) in the dark for 30 min at 30° C. Cells were washed two times with Hanks Balanced Salt Solution (I-SS) (Cell Gro) containing 1% FBS. Cells were then surface stained with APC-anti-CD4 Ab (Pharmingen) for 20 min at RT, washed 2× with 1-113SS/1% FBS, and incubated on ice with 5 ug anti-CD3Σ (2C11) antibody. Cells were then cross-linked with goat anti-hamster Ig (Jackson) at 37° C., and calcium flux was measured using a FACS Calibur. Data was analyzed using FlowJo software.

### Example 8

### Actin Polymerization Assays

For actin polymerization studies, purified CD44 T cells or retrovirally infected CD4⁺ T cells were incubated with anti-hamster CD3 Ab (2C11, 1 μg/ml) for 30 milt on ice. After washing, the primary antibodies were cross-linked using goat anti-hamster Ig (2 μg/ml) for 5 min at 37° C. The cells were then fixed in 3.7% formaldehyde, washed and permeabilized in 0.1% Triton X-100/PBS. After washing, the cells were incubated with FITC-conjugated Phalloidin (Sigma) for 30 min. For retrovirally infected T cells, cells were incubated with biotin-conjugated Phalloidin (Molecular Probes) followed by streptavidin-PE (Pharrningeit) staining. Cells were washed three tunes with PBS and analyzed by flow cytometry.

### Example 9

### Retroviral Transduction

The retroviral constructs for wild-type IBP and an IBP deletion mutant lacking amino acids 313-631 (IBP A313-631) were generated by cloning either the entire coding region of the IBP cDNA or its appropriate coding segment, respectively, into the pGCIRES-YFP retroviral vector (34). 293 T cells were plated at 2×10⁶ per dish on 10 cm plates in DMENI with 10% F13S, and grown for 24 h. The cells were then co-transfected with the retroviral plasmids together with the retroviral packaging vector pCL-Eco by the calcium phosphate/DNA precipitation method. After an overnight incubation, the medium was replaced with fresh DMEM plus 10% FBS. After 24 h, the retroviral supernatants were harvested from the transfected 293T cells, supplemented with 8 μg/ml polybrene, and then used for infection on naive CD4⁺ T cells that had been stimulated for 24 h with 10 μg/ml anti-CD3 Ab, 10 μg/ml anti-CD28 Ab, and 10 ng/ml recombinant IL-2. The naive CD4⁺ T cells were centrifuged with the retroviral supernatants for 1.5 hr at 1800 rpm at 32° C., and then incubated overnight at 37° C., followed by an additional retroviral infection. After the infections, the T cells were cultured in fresh medium containing 10 ng/ml recombinant IL-2. On day 5 after initial stimulation, functional assays were then performed. For intracellular cytokine staining experiments, CD4⁺ T cells were cultured with I ltg/rnt anti-CD3 Ab or 50 μg/ml PMA and 1 μM lonomycin in the presence of Golgi Stop (BD Pharmingen) for 4 h. Cells were then harvested, fixed, permeabilized, and stained with APC-conjugated anti-IFN-γ Ab (BD Pharmingen) for 30 mire, Cells were washed, resuspended in Igo BSA/PBS, and analyzed using FACS Calibur using Flowlo software (Tree Star, San Carlos, Calif.).

### Example 10

### Lymphoid Development is not Affected by Lack of IBP

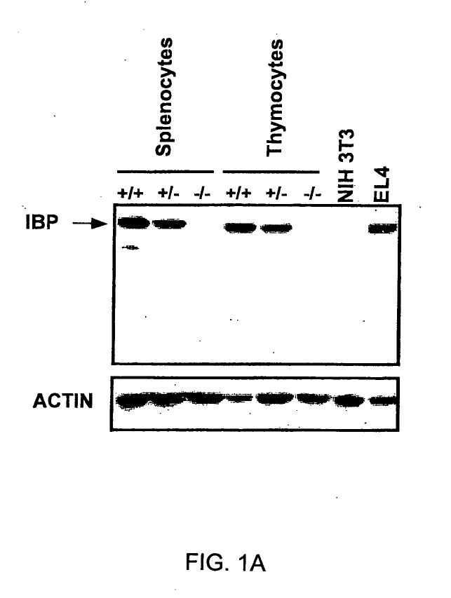

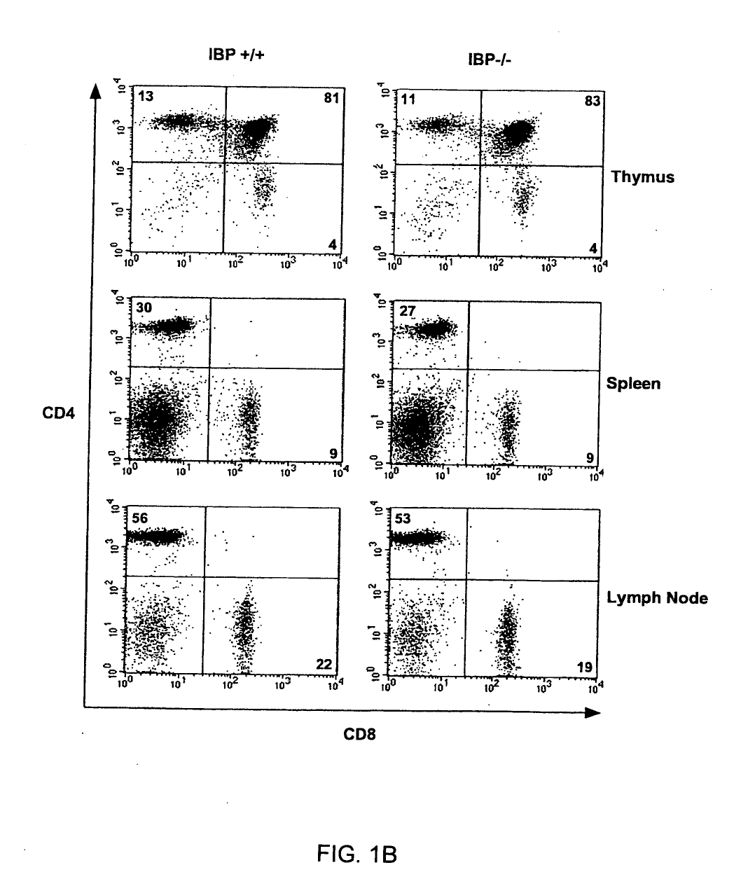

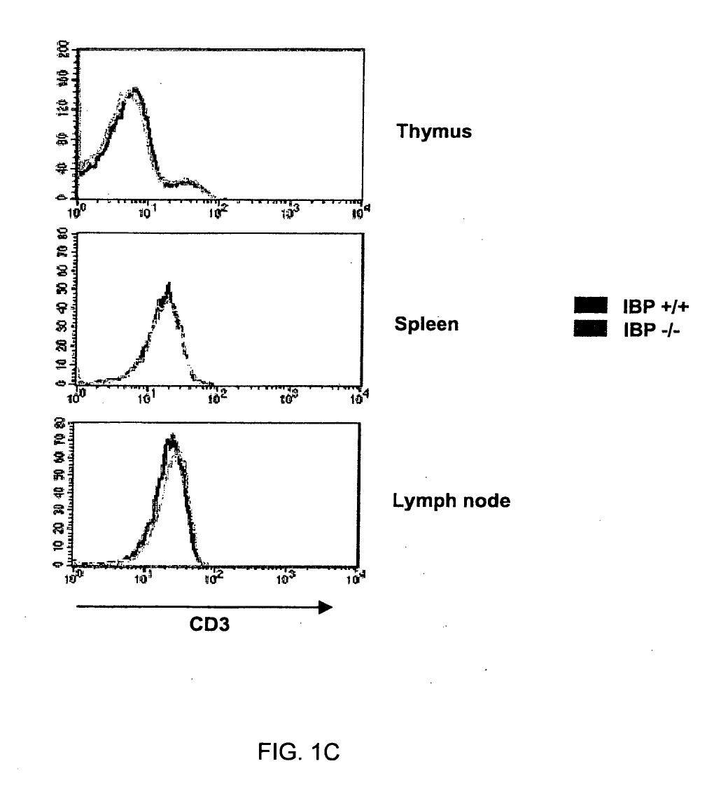

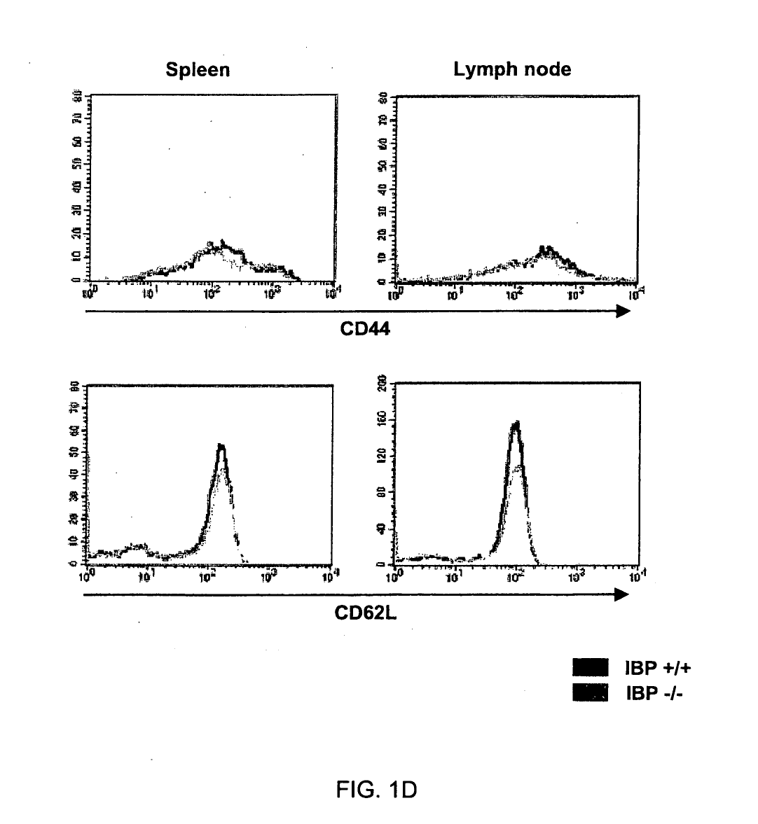

To elucidate the role of IBP in vivo, IBP-deficient mice were generated utilizing a gene trapping strategy (32). Integration of the gene trapping construct occurred in the 1^{st} intron of the IBP gene resulting in the complete absence of IBP expression as demonstrated by Western blot analysis with an antibody directed against the carboxy-terminus of IBP (FIG. 1A) (26). IBP-deficient mice were born at the expected Mendelian frequency. They were viable, fertile and phenotypically normal. An analysis of the cellularity and surface phenotypic markers in mutant and control mice revealed that the lymphoid organ sizes were similar and that there were no obvious T or B cell developmental defects (Table t and FIG. 1B). Furthermore, IBP^{-/-} mice did not exhibit any abnormalities in CD3 or LFAI expression levels (FIG. 1C and data not shown) and contained normal numbers of naYve and memory CD4⁺ T cells as compared to IBPr′ mice (Fig. ID). These results thus indicate that the absence of IBP does not lead to any obvious detects in lymphocyte development.

### Example 11

### Lack of IBP Leads to Selective Impairments in CD4⁺T Cell Effector Function

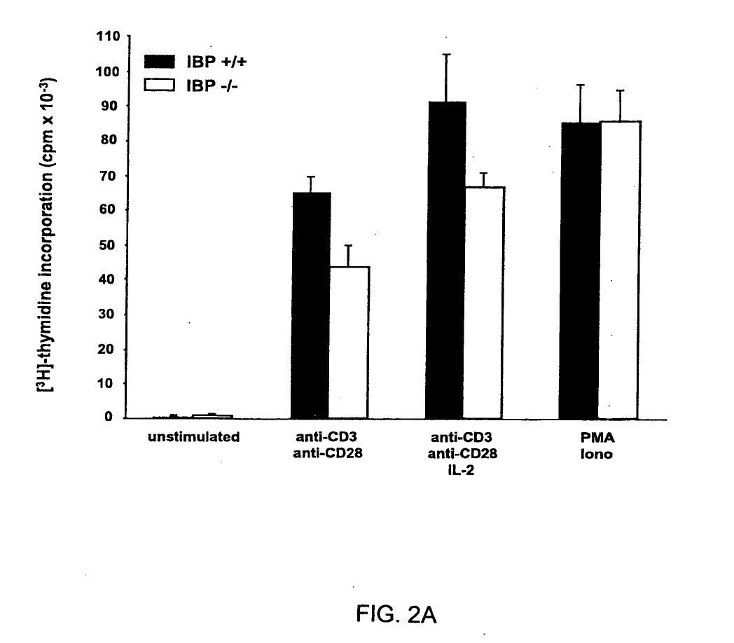

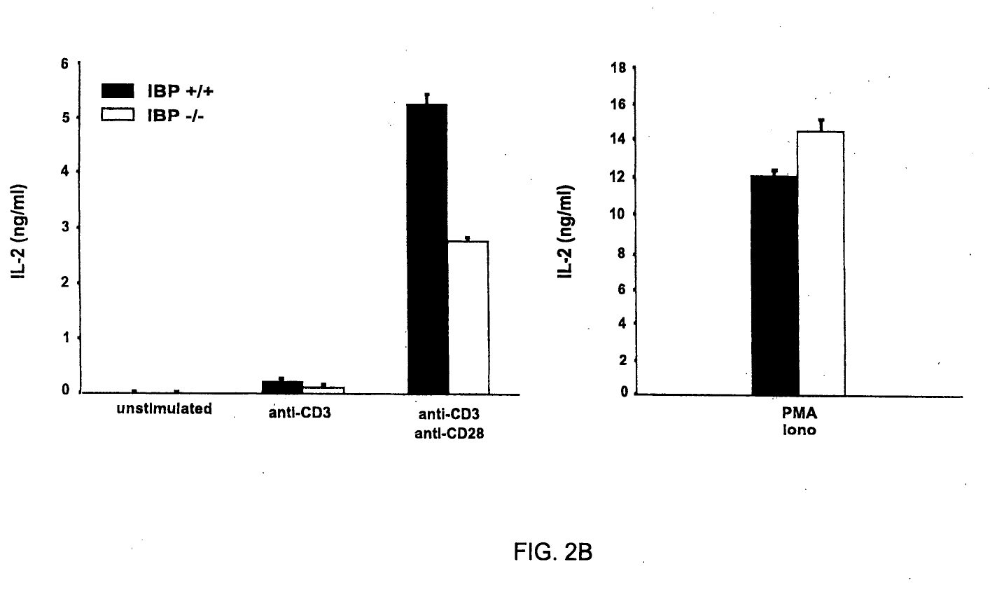

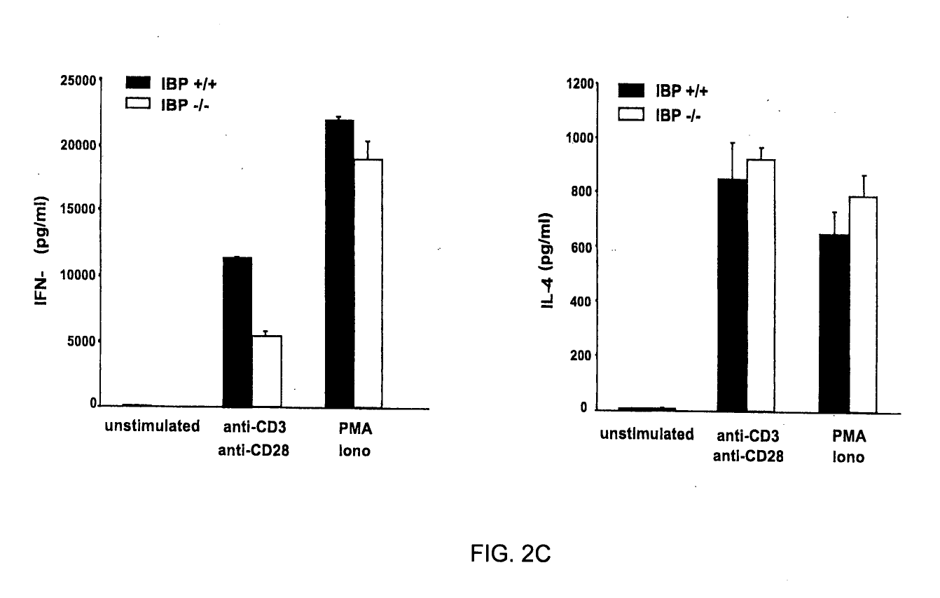

Given the high level of expression of IBP in the T cell zone of peripheral lymphoid organs (26), the inventors next examined whether lack of IBP affects the function of peripheral T cells. Purified CD4⁺ T cells were stimulated with plate-bound anti-CD3 monoclonal antibody in the presence of anti-CD3 stimulation. The proliferation of IBP^{-/-} T cells was reduced as compared to IBP^{+/+} T cells (FIG. 2A). Addition of 1L-2 only partially rescued the proliferative defect exhibited by the IBP-deficient T cells. Bypassing proximal TCR signaling events by stimulating IBP^{-/-} T cells with phorbol 12-myristate 13-acetate (PMA) and calcium ionophore (iono nycin), however, led to proliferative responses similar to those of control T cells. These results thus indicate that optimal T cell proliferative responses require the presence of IBP.

The induction of CD69 and CD25 (IL-2 receptor a chain), two surface antigens known to be upregulated by T cell stimulation, was also moderately impaired in IBP-deficient T cells (data not shown). These defects were accompanied by a reduction in the ability of IBP-deficient T cells to produce IL-2 upon TCR stimulation (FIG. 2C). Stimulating the cells with PMA and ionomycin again rescued the defective IL-2 production exhibited by the IBP″ T cells. Interestingly, the production of IFN-γ was also impaired by the lack of IBP (FIG. 2C, left panel), while IL-4 synthesis was comparable to that of control T cells (FIG. 2C, right panel). These data thus demonstrate that IBP is required for proximal TCR. signaling events necessary to achieve full effector function.

### Example 12

### TCR-Driven but not Cytokine-Driven TH Differentiation is Impaired in the Absence of IBP

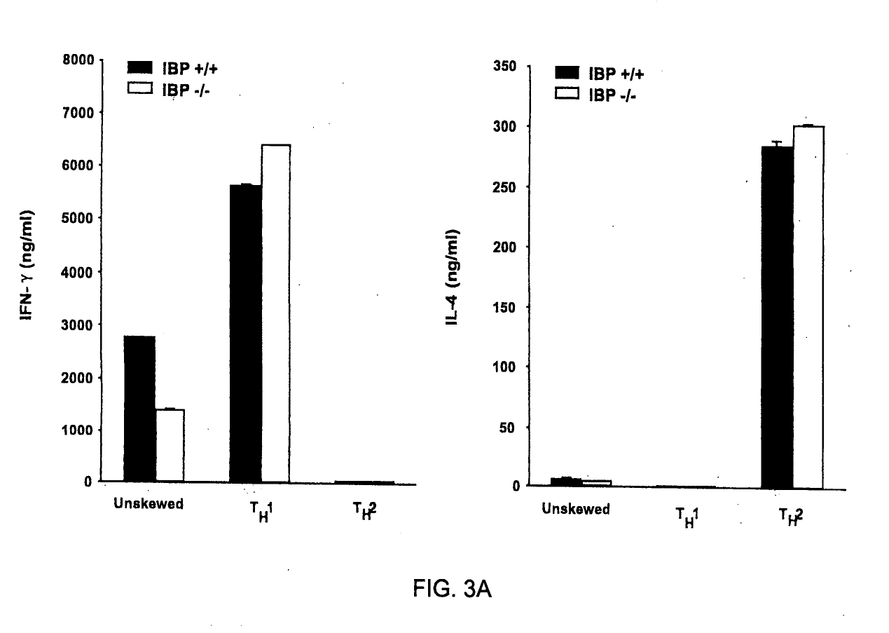

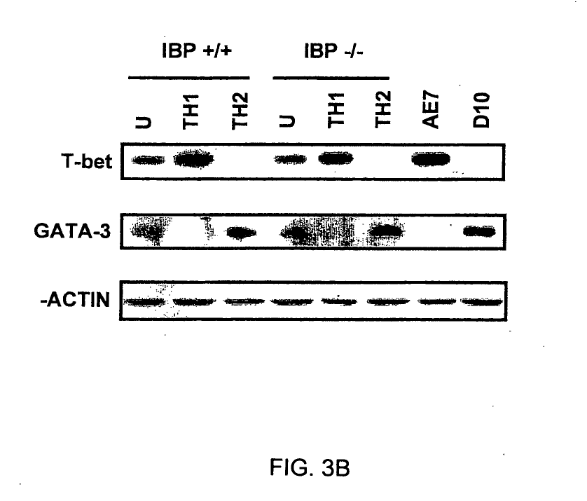

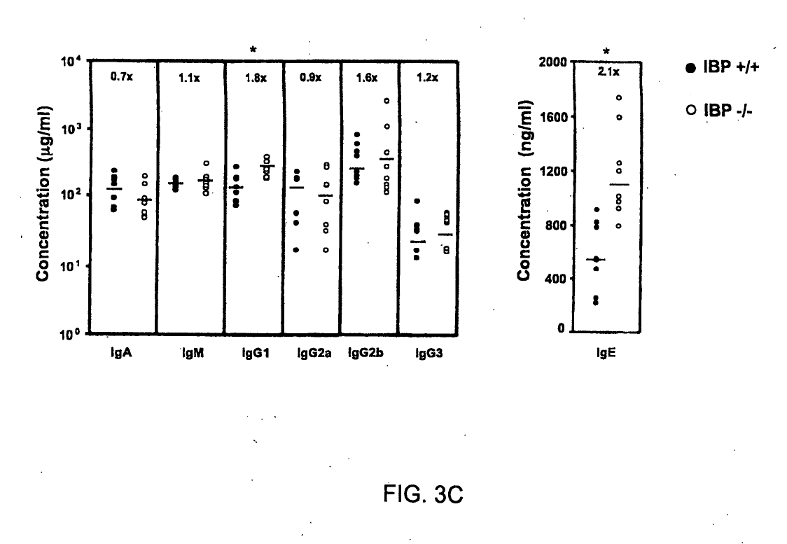

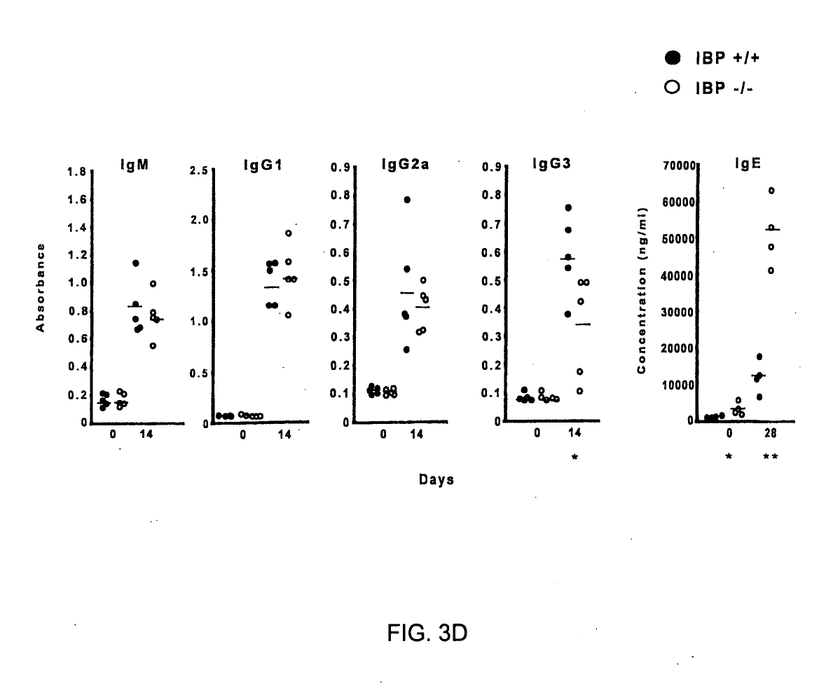

To investigate whether lack of IBP leads to global defects in the ability of T cells to produce TH1-type cytokines, naive CD4⁺ T cells from IBP^{+/+} and IBP^{-/-} mice were primed either in the absence of cytokines or in the presence of cytokines that would skew them toward a TH1 or a TH2 differentiation pathway. After 7 days, cells cultured under each of the three conditions were restimulated with anti-CD3 and cytokine production was assessed by ELISA (FIG. 3A). Consistent with the inventors' earlier observations, IBP^{-/-} T cells cultured in the absence of cytokines exhibited defective IFN-γ production but maintained a relatively normal ability to synthesize.

Priming of IBP^{-/-} T cells with cytokines known to drive TH1 differentiation partially or even completely rescued the defective production of IFN-γ by IBP T cells. Exposure of IBP^{-/-} T cells to TH2 skewing conditions also failed to reveal any striking defects in IL-4 production. Consistent with these findings, up-regulation of T-bet and GATA-3, two critical regulators of cytokine driven TH 1 and TH2 differentiation respectively, were similar in TH1 and TH2 cells derived from control and IBP^{-/-} mice (FIG. 3B). These data indicate that IBP is crucial for TCR mediated IFN-γ production. In the presence of TH1-skewing cytokines, however, the requirement for IBP is less stringent.

To determine whether the altered pattern of cytokine production that the inventors detected in vitro was reflected into a skewing of immune responses in vivo, the inventors next examined the serum immunoglobulin levels of wild-type and D3P-deficient mice. IBP-deficient mice displayed increases in basal serum levels of IgG₁ and IgE, while the levels of other isotypes were essentially comparable to those of wild-type mice (FIG. 3C). Furthermore, IBP^{-/-} mice immunized with the T cell-dependent (TD) antigen NP-KLH exhibited profound increases in the production of IgE (FIG. 3D). Thus, lack of IBP results in enhanced TH2-type humoral responses to T-dependent antigens.

### Example 13

### TCR-Mediated Activation of Protein Tyrosine Kinases in IBP Deficient Mice

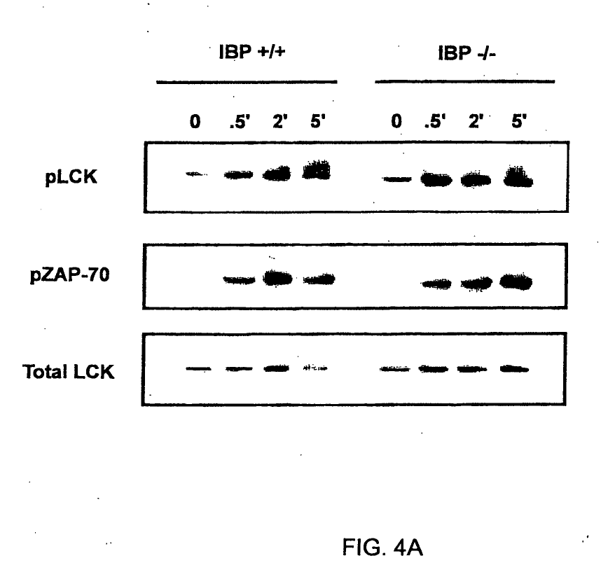

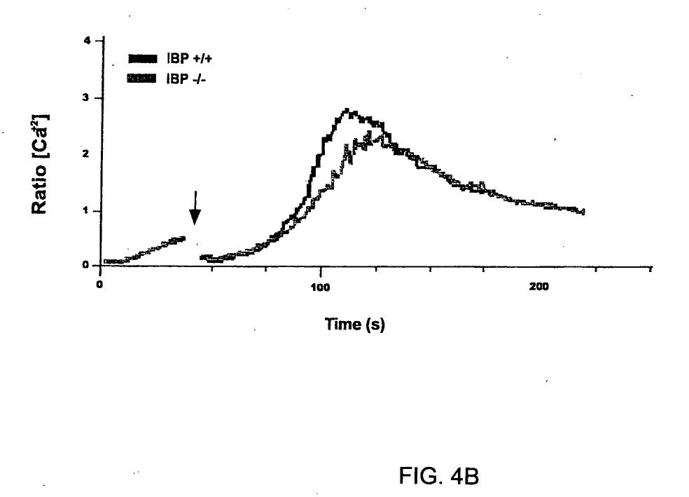

The rapid activation of IBP in response to TCR signaling coupled with the finding that the functional defects observed in the IBP^{-/-} T cells could be rescued by stimuli that bypass the proximal signaling steps prompted us to directly investigate whether IBP deficiency affects TCR signaling. One of the earliest signaling events triggered by engagement of the TCR is the activation of the Src kinases, Lck and Fyn (1, 2). This step is crucial for the recruitment and activation of another kinase, ZAP-70, which is then followed by the phosphorylation of additional targets. The inventors first examined whether lack of IBP affects the TCR-mediated activation of Lck by employing an antibody directed against Y394, whose phosphorylation status correlates with Lck activation (33). As shown in FIG. 4A (upper panel), phosphorylation of Lck proceeded to a similar extent in both wt and IBP mutant T cells. Similarly, the TCR-mediated activation of ZAP-70, as reflected by the phosphorylation of tyrosine 319, was also largely unaffected by the lack of IBP (FIG. 4A, middle panel). These results this indicate that the absence of IBP does not affect the TCR-mediated activation of protein tyrosine kinases.

Calcium fluxes are rapidly triggered following the initial TCR-evoked PTK activation (1, 35). Interestingly, T cells from mice deficient in members of another family of GEFs for Rho GTPases, the Vav family, display severe impairments in TCR-mediated calcium mobilization (21-23). To determine whether lack of 1BP also affects Ca2⁺ flux in T cells, the inventors employed flow cytometry to analyze calcium mobilization in wt and mutant splenic T cells upon CD3 crosslinking. As shown in FIG. 4B, mutant T cells were able to achieve cytosolic Ca′Y levels comparable to those of wild-type T cells although IBP deficient T cells consistently displayed a slightly longer lag time to the onset of Ca⁺2 signal. Consistent with these results, mobilization of the calcium-sensitive transcription factor nuclear factor of activated T cells (NEAT) proceeded normally in IBP mutant T cells (data not shown). Thus, in contrast to the Vav family of proteins, IBP is not required for TCR-induced Ca2⁺ signaling.

### Example 14

### Defective IH RKL/ERK2 Activation in the Absence of 1BP

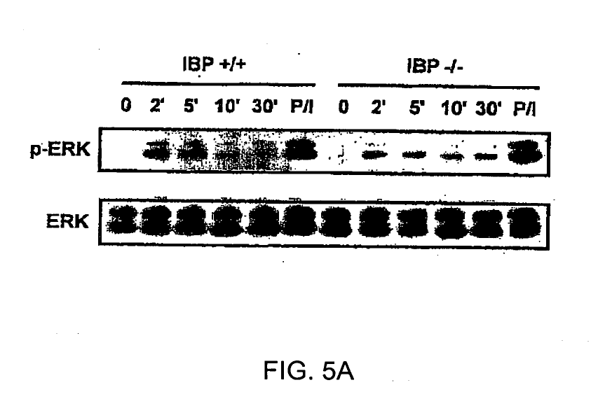

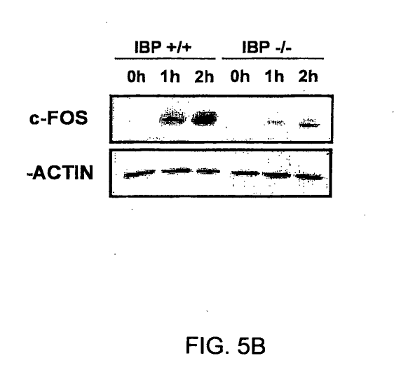

In addition to calcium mobilization, the initial wave of protein tyrosine kinase activity stimulated by TCR crosslinking is followed by the activation of additional downstream effector kinases, most notably ERKI/2, members of the mitogen-activated protein kinase (MAPK) family (1, 35). Activation of ERKI/2 subsequently mediates the induction of immediate early genes like c-Fos (36). An examination of the activation of ERK1/2 demonstrated that TCR-mediated ERKI/2 activation was markedly reduced in IBP-deficient T cells as compared to wild-type T cells (FIG. 5A). IBP-deficient T cells, however, were able to activate ERKI/ERK2 to levels similar to wild-type cells when stimulated with FMA (FIG. 5A). Consistent with these findings, I13P-deficient T cells also exhibited impairments in the upregulation of the immediate early gene, c-Fos (FIG. 5B). Thus, lack of IBP leads to selective defects in the downstream signaling machinery required for the amplification of signals emanating from the TCR.

### Example 15

### TCR-Induced Actin Polymerization and Immunological Synapse Formation are Defective in HIP-Deficient T Cells

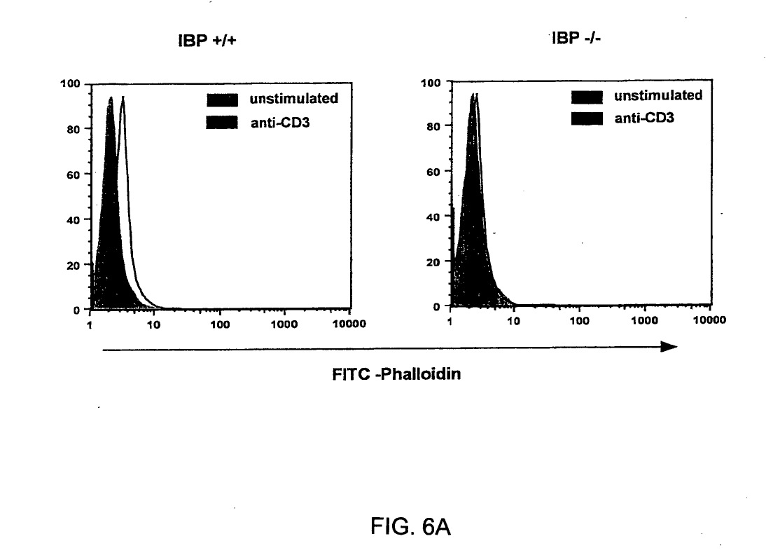

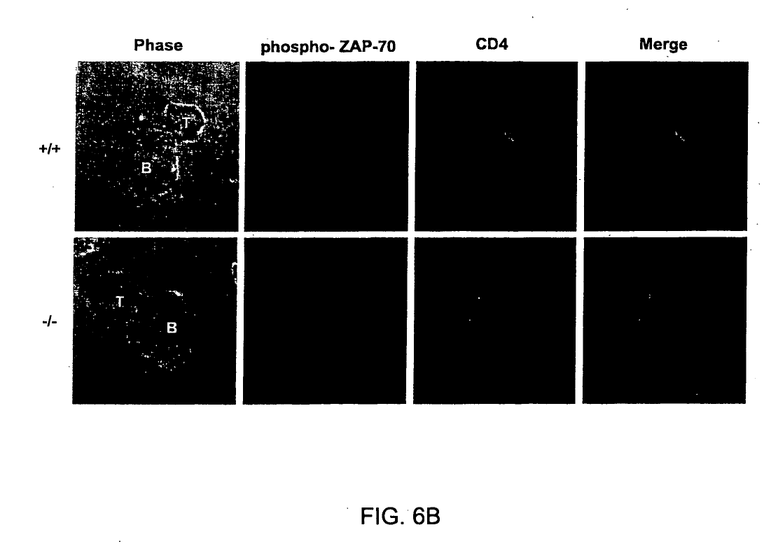

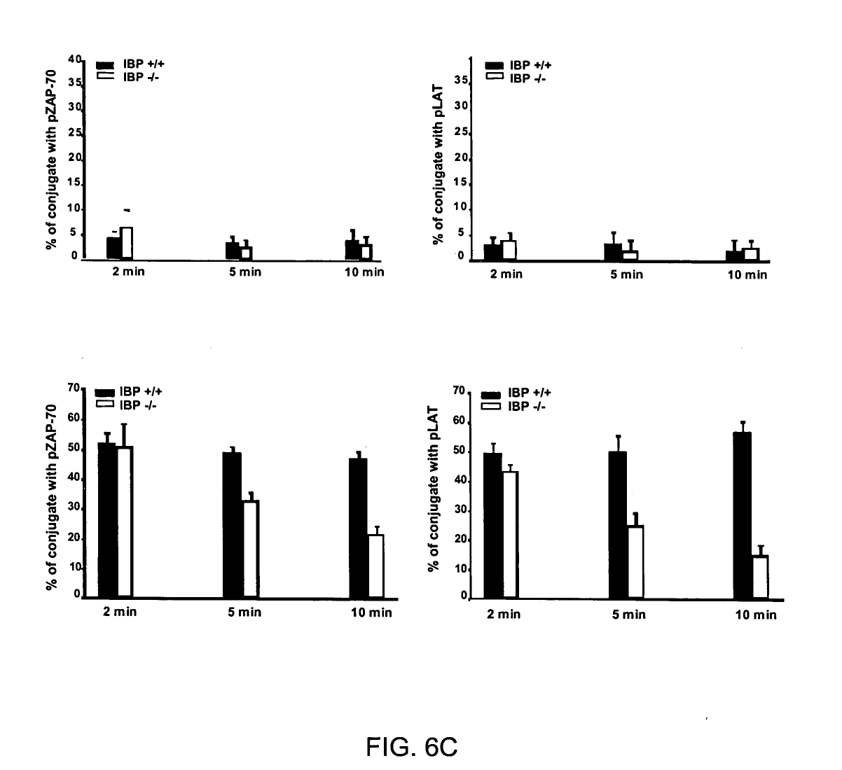

Optimal transmission of TCR-mediated signals requires actin cytoskeletal reorganization and assembly of the immunological synapse (6-11). The inventors' previous studies had indicated that, upon TCR engagement, IBP rclocalizes to the synapse and activates Rho GTPases, key mediators of cytoskeletat. dynamics (30). These findings led us to investigate whether IBP is involved in the regulation of T cell cytoskeletal remodeling. The effect of TCR stimulation on actin polymerization was analyzed using FITC-labeled Phalloidin (FIG. 6A). In the absence of stimulation, wild-type and mutant T cells contained similar amounts of F-actin. Upon TCR stimulation, however, wild-type T cells demonstrated an increase in F-actin content while 1BP-deficient T cells failed to do so. The inventors next investigated whether IBP is required for the appropriate assembly of the immunological synapse. Purified CD4⁺ T cells from wild-type and IBP-deficient mice were incubated with APCs in the presence or absence of superantigen (SEF) (FIGS. 6B and 6C). Conjugate formation was then assessed by determining the recruitment of a phosphorylated form of ZAP-70 to the T cell-APC interface. The initial recruitment of phosphorylated ZAP-70 to the synapse was similar in IBP^{+/+} and IBP^{-/-} T cells. However, at later time points, IBP-deficient T cells displayed a markedly decreased accumulation of phosphorylated ZAP-70 at the T cell-APC interface (FIGS. 6B and 6C). A similar pattern was observed when the recruitment of a phosphorylated form of LAT was examined (FIG. 6C). Taken together, these data suggest that IBP is crucial for TCR-mediated cytoskeletal reorganization and that, in its absence, the immunological synapse cannot be properly assembled.

### Example 16

### Retroviral Expression Of IBP Deficient T Cells with WT IBP but not with an IBP Mutant Lacking the DH Domain Rescues Actin Polymerization and IFN-γ Production

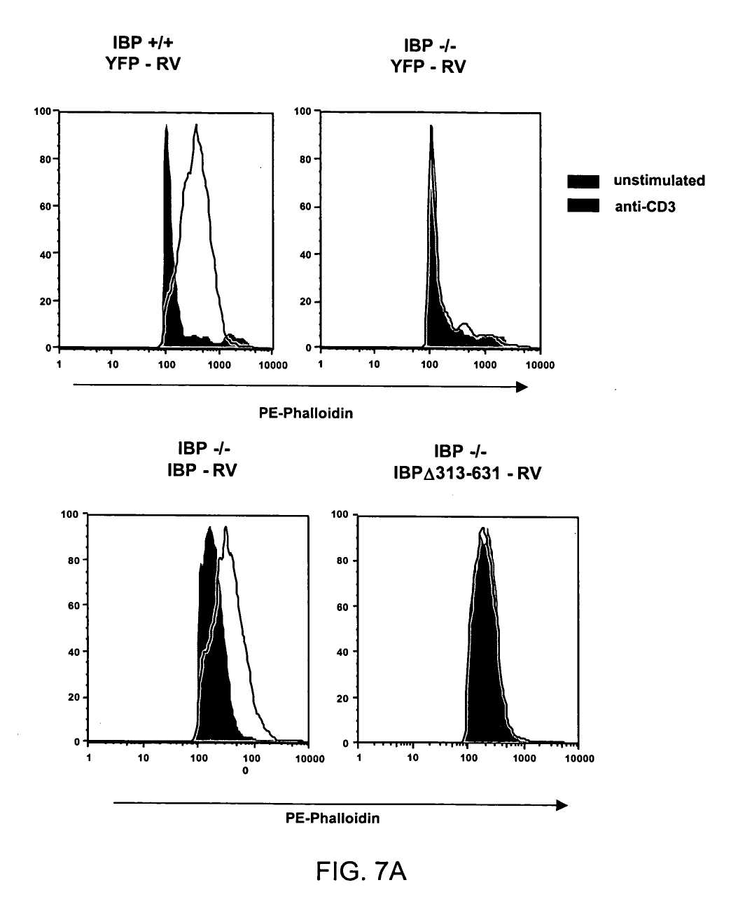

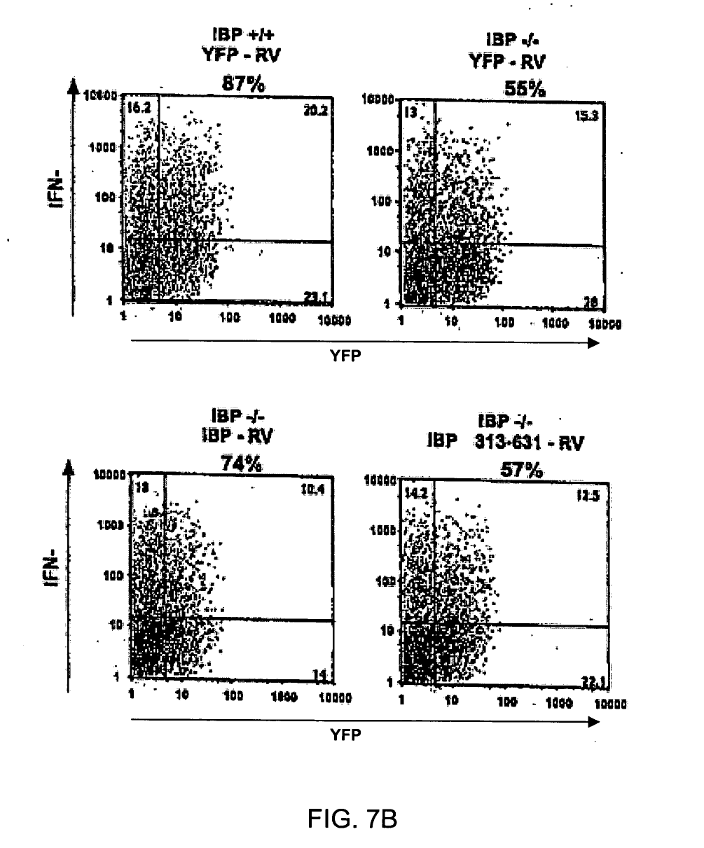

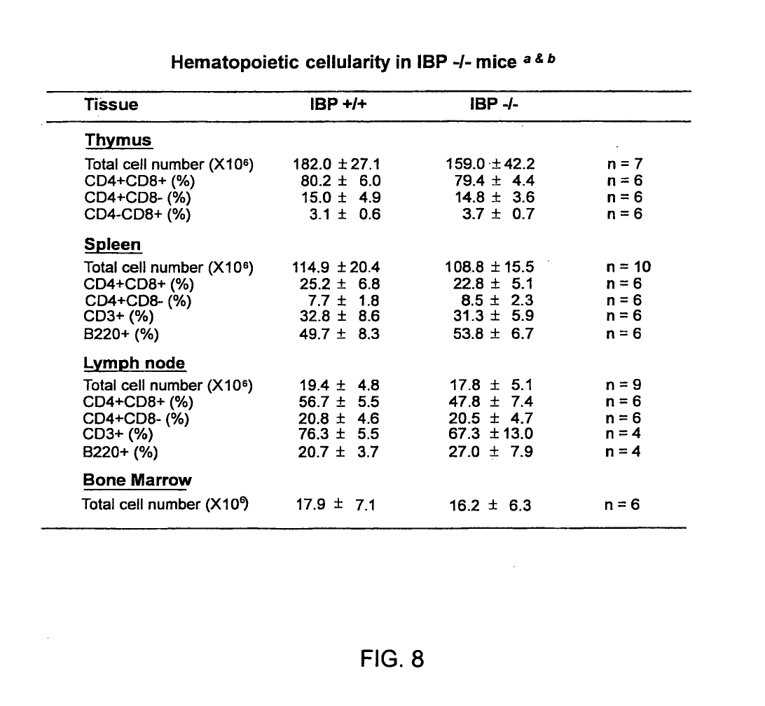

The inventors have previously demonstrated that the GEF activity of IBP maps to a DH domain located at its carboxy-terminus (30), To explore the mechanisms responsible for the defects observed in the IBP mutant T cells the inventors therefore employed a retroviral transduction system to introduce either wild-type IBP (IBP-RV) or an IBP mutant lacking the DH domain ONO] 3-631-RV) into IBP^{-/-} primary T cells. As shown in FIG. 7A, reconstitution of wt IBP expression into IBP deficient T cells was able to restore actin polymerization to levels comparable to those obtained by wt T cells. In striking contrast, IBP deficient T cells infected with IBPA313-631-RV were still incapable to polymerize actin in response to TCR stimulation. In order to determine whether the GEF activity of 11W is also required for the effector functions of T cells, T cells reconstituted with the different IBP constructs were next assayed by intracellular cytokine staining for their ability to produce IFN-γ upon TCR stimulation (FIG. 7B). Infection of IBP. T cells with IBPRV, but not with 1BRA313-631-RV, increased the percentage of IFN-γ producing cells to levels equivalent to those of IBP^{+/+} T cells. Taken together these data suggest that the GEF activity of IBP is required for TCR-mediated cytoskeletal reorganization and acquisition of full T cell effector function.

Discussed below are results obtained by the inventors in connection with the experiments of Examples 1-21:

Analysis of MP-deficient mice demonstrates that IBP plays a unique role in mature T cell function. IBP-deficient T cells exhibit selective impairments in the TCR-mediated production of IL-2 and IFN-γ but not in that of IL-4. Furthermore, IBP deficient mice display enhanced levels of serum IgE indicating that humoral responses in these mice are preferentially skewed toward a TH2 phenotype. The functional impairments exhibited by 1BP deficient mice are associated with suboptimal transmission of TCR-mediated signals, impaired cytoskeletal remodeling, and defective synapse formation. Mechanistically, reintroduction of full-length IBP, but not of an IBP mutant lacking its GEF activity, rescued the defective responses of the IBP^{-/-}T cells indicating that activation of Rho GTPases by IBP is crucial for the full execution of the activation program of T cells.

The finding that IBP plays a key role in the production of IFN-γ is consistent with previous studies that have implicated Rac2, a hentatopoietic restricted Rho GTPase. in the control of IFN-γ production (36). Exposure to Till-skewing cytokines like IL-12 was able to largely rescue the defects in IFN-γ production exhibited by the IBP deficient T cells suggesting that the requirement for IBP is less stringent when a T cell encounters an antigen in the presence of strong polarizing conditions.

In agreement with the finding that lack of IBP affects the production of THI-type cytokines to a greater extent than TH2-type cytokines, absence of IBP led to enhanced production of isotypes usually associated with TH2 responses. Indeed, lack of IBP resulted in markedly increased IgE levels upon immunization with T-dependent antigens, which is in striking contrast with the impaired IgE production exhibited by SWAP-70 deficient mice (28). Surprisingly, purified IBP^{-/-} B cells stimulated in vitro with anti-CD40 Ab and IL-4 did not produce increased levels of IgE (unpublished observations) suggesting that the contrasting effects of these two family members on IgE production are not simply due to opposing regulatory actions on B cells. The profound deregulation of IgE responses displayed by the IBP deficient mice is however unlikely to be solely due to the defective ability of T cells to produce TH1 cytokines and may include contributions from additional cellular compartments. In particular, since IBP is expressed in dendritic cells (unpublished observations), alterations in dendritic cell development and/or function may participate in the enhanced IgE responses exhibited by the IBP mice.

Previous studies have shown that potent TCR signals are required for IFN-γ production while weaker TCR signals suffice to drive IL-4 synthesis (3). Thus, the selective defects in the production of IFN-γ displayed by IBP deficient T cells are likely to result from the suboptimal transmission of TCR signals that occurs in the absence of IBP. In particular, given that differential activation of ERK1/ERK2 has been associated with distinct cytokine profiles (37), the inability of IBP mutant T cells to strongly activate these MAPKs and the resulting defects in immediate early gene production may be directly linked to these events. Indeed, members of the AP-I family of transcription factors have previously been implicated in the regulation of IFN-γ gene transcription (38). The mechanism by which IBP controls this pathway is likely to require its GEF activity since activation of MAPK is one of the crucial downstream effector pathways controlled by Rho GTPases.

TCR-mediated actin polymerization and synapse assembly were profoundly affected by the lack of IBP. In particular, these studies show that the effects of IBP on T cell cytoskeletal reorganization required its GEF activity since they could not be rescued by expression of an IBP mutant lacking its GEF domain. IBP deficient T cells were unable to properly sustain the formation of the immunological synapse. Previous studies have shown that the full achievement of T cell effector potential requires sustained synapse assembly, which, in turn, necessitates optimal T cell stimulation (39). It has been furthermore shown that while suboptimal stimulatory conditions can lead to an early phase of actin accumulation at the T cell-APC interface, sustained actin accumulation and long-range actin translocation only occur under optimal stimulatory conditions (40). IBP may thus be one of the critical components of the TCR signaling machinery that controls the sustained actin dynamics required for the formation of a complete immunological synapse.

Similarly to the IBP deficient mice, mice lacking Vav proteins also display defects in cytoskeletal dynamics (41, 43) as well as alterations in ERK activation (44, 45) suggesting that the actions of both GEFs are required for these processes. Given that Rho GTPases can control both the activation of ERKI/2 as well as cytoskeletal reorganization via multiple mechanisms (14-16, 19), it will be important to determine whether these different classes of GEFs cooperate in the control of a specific regulatory step or target different steps in the complex cascades required to optimally activate these processes. In contrast to IBP-deficient mice, Vav deficiency also results in severe impairments in T cell development as well as profound defects in TCR-induced calcium fluxes (41, 42, 44). The broader effects exerted by Vav may be due to its ability to mediate not just GEF-dependent but also GEE-independent actions (46). Alternatively, different subsets of Rho GTPases may serve as the physiologic targets of these different GEFs. In this regard it is interesting to note that while genetic manipulations of Rac1 expression/function exert profound effects on T cell development, manipulations of Rac2 primarily affect the function of peripheral T cells (17, 18).

Collectively, this data indicate that IBP uniquely links the TCR to the acquisition of full T cell effector function and that its recruitment/activation upon TCR engagement may be critical for the transmission of high potency TCR signals. The defective T cell responsiveness observed in the absence of IBP is likely to result from its ability to control both TCR-mediated signaling as well as cytoskeletal reorganization via activation of Rho GTPases. The ability of IBP to control T cell responsiveness coupled with its location on human chromosome 6 just centromeric to the MHC locus (26) may thus warrant an investigation of the role of this novel molecule in immune-mediated pathophysiotogical states like autoimmune disorders and allergic conditions.

### Example 17

### Cell Cultures and Transfections

The various Jurkat (human T cell leukemia) cell lines, including JE6-1 (wild-type), J.CaM1.6 (Lck-deficient), and J-TAg (SV40 large T-antigen-transfected) cell lines, were obtained from American Type Culture Collection (ATCC, Manassas, Va.). All the cell lines were grown in RPMI 1640 medium supplemented with 10% heat-inactivated fetal calf serum (Atlanta Biologicals, Inc.), 2 mM L-glutamine, 10 mM HEPES, and antibiotics. 293T (a human embryonic kidney cell line) cells were a kind gift of Dr. Chris Schindler, Columbia University, New York, N.Y. and were grown in Dulbecco's modified Eagle's medium supplemented with 10% fetal calf serum. COS-7 cells (a generous gift of Dr. Steven Greenberg, Columbia University, New York, N.Y.) were grown in RPMI 1640 medium supplemented with 10% fetal calf serum. Raji cells were obtained from Dr. Raphael Clynes (Columbia University) and were grown in RPMI 1640 medium supplemented with 10% fetal bovine serum. Jurkat T cells were washed with serum-free RPMI 1640 medium, serum-starved for 5 h, and then stimulated at 37° C. for the indicated time periods with a mouse anti-human CD3 mAb (clone OKT3) followed by cross-linking with a secondary goat anti-mouse Ig antibody, as described previously (23). For expression of recombinant proteins, 293T cells and COS-7 cells were transfected with expression plasmids by the calcium phosphate/DNA precipitation method or the SuperFect transfection reagent (Qiagen, Inc.), respectively. After 24 h of incubation, the transfected cells were harvested for cell extract preparation.

### Example 18

### DNA Constructs

The full-length wild-type human IBP expression plasmids (pCEP4-HA-IBP and pIRES2-EGFP-HA-IBP) were constructed by cloning the entire coding region of the human IBP cDNA, fused in-frame with a hemagglutinin (HA) epitope coding sequence at its 5′ terminus, into the pCEP4 expression vector (Invitrogen) or pIRES2-EGFP bicistronic expression vector (Clontech), respectively. The point mutations Y210F or R236C in the full-length IBP were introduced by the site-directed mutagenesis method (Stratagene) and confirmed by DNA sequencing. Various deletion mutants of human IBP were generated by PCR using appropriate primers and confirmed by DNA sequencing. For preparation of various glutathione S-transferase (GST)-IBP fusion proteins, the corresponding GST-IBP expression plasmids were generated by cloning either the entire coding sequence of the human IBP cDNA or its appropriate segments, in-frame, into the pGEX-KG Escherichia coli expression vector (Amersham Biosciences). The in-frame junction in the GST-IBP fusion constructs was confirmed by DNA sequencing. The constitutively active Lck(Y505F) expression construct in pcDNA3 mammalian expression vector (24) was a kind gift of Dr. Jerry Siu.

### Example 19

### Protein Purification, Antibodies, Cell Extracts, and Protein Assays

GST-IBP fusion proteins were expressed in E. coli DH5a and affinity-purified on glutathione-agarose beads (Sigma), as described previously (25). HA epitope-tagged IBP (full-length wild-type) was expressed in 293T cells and affinity-purified on immobilized anti-HA monoclonal antibody (clone 3F10; anti-HA affinity matrix; Roche Applied Science) according to the procedures recommended by the manufacturer. The polyclonal anti-IBP antibody was generated by immunizing rabbits with purified GST-IBP (amino acids 410-631) fusion protein (Covance, Inc., Princeton, N.J.). This GST fusion protein contains a portion of the human IBP protein, which is least homologous to SWAP-70 and thus minimizes cross-reactivity of the antibody with SWAP-70 (21). The anti-IBP antibody was utilized at 1:1000 in Western blotting and at 1:200 in the immunofluorescence experiments. An anti-phosphotyrosine monoclonal antibody (clone 4G10) was obtained from Upstate Biotechnology. The rat monoclonal antibody against HA epitope (clone 3F10) was purchased from Roche Applied Science. The PKC-0 and P-actin antibodies were purchased from Santa Cruz Biotechnology, Inc.

Whole cell extracts were prepared as described previously (26). Cell lysates were immunoprecipitated with an anti-IBP antibody or anti-HA mAb (clone 3F10) as described previously (26). The immunoprecipitates were resolved by 7% SDS-PAGE. The gel was transferred to a nitrocellulose membrane and then immunoblotted with an anti-phosphotyrosine antibody (4G10) or the anti-IBP antiserum. The bands were visualized by ECL (Amersham Biosciences). Pull-down assays with phosphoinositide analogue beads (Echelon Research Laboratories Inc.) were performed according to the manufacturer's instructions.

Lck-mediated tyrosine phosphorylation of IBP was assessed by in vitro Lck kinase assay using purified Lck kinase (Upstate Biotechnology) and either purified HA epitope-tagged IBP (wild-type) or immunoprecipitated recombinant IBP proteins (wild-type or Y210F mutant) according to previously described protocols (27). Briefly, purified HA-IBP (˜150 ng) or immunoprecipitates of recombinant IBP proteins were incubated with 5 units of purified Lck in 30 μl of 1× kinase buffer (20 mM HEPES, pH 7.4, 100 mM NaCl, 5 mM MnCl2, 10 mM MgCl₂, 50 μM Na3VO4, 1 mM dithiothreitol) containing 1 μM cold ATP and 10 μCi of [γ-³²P]ATP for 30 min at 30° C. The reactions were terminated by adding SDS-PAGE sample buffer and boiling. The reaction samples were resolved on a 7% SDS-polyacrylamide gel. The gel was fixed, soaked in 1 N KOH at 55° C. for 2 h, refixed, dried, and then autoradiographed to visualize tyrosine-phosphorylated products.

### Example 20

### In Vivo RAC1/CDC42 Activation Assay and In Vitro GDP Release Assay

For the in vivo activation assays for Rac1 and Cdc42, COS-7 cells were transiently transfected with an appropriate expression vector for either wild-type HA-IBP or various IBP deletion mutants. The cells were washed with ice-cold phosphate-buffered saline containing 5 mM MgCl2 and then lysed in 1× lysis buffer (25 mM HEPES, pH 7.5, 150 mM NaCl, 1% Nonidet P-40, 10 mM MgCl2, 1 mM EDTA, 10% glycerol, 2 mM dithiothreitol, 1 mM Na3VO4, protease inhibitors). Activated GTP-bound Rac1 (or Cdc42) was affinity-precipitated from the cell lysates by using GST-PAK1 PBD (p21 Rac/Cdc42-binding domain) fusion protein immobilized onto glutathione-agarose beads (Upstate Biotechnology) according to the manufacturer's instructions. The precipitated Rac1-GTP or Cdc42-GTP was resolved by 12.5% SDS-PAGE and then visualized by Western blot analysis with either an anti-Rac1 antibody (Upstate Biotechnology) or an anti-Cdc42 antibody (Transduction Laboratories), respectively.

The in vitro GDP release assays were carried out by filter-binding method as described previously (28-31). To prepare [3H]GDP-loaded GTPases, 40 pmol of bacterially expressed and purified GST-Rac1, GST-Cdc42, or His-RhoA (Calbiochem-Novabiochem) was incubated with 1 μM [3H]GDP in 100 μl of a binding buffer (10 mM HEPES, pH 7.5, 100 mM NaCl, 5 mM MgCl2, 1 mM EGTA, 1 mM dithiothreitol, 50 μg/ml bovine serum albumin) for 60 min at 30° C. The nucleotide exchange reaction was initiated by adding 100 μM nonradioactive GTP and 4 pmol of purified GST alone or GST-IBP fusion proteins (or when appropriate, 4 pmol of purified wild-type HA-IBP either non-phosphorylated or in vitro phosphorylated by purified Lck and cold ATP) to the [3H]GDP-loaded GTPase and then equally splitting and incubating the reaction mixture for the indicated time periods at room temperature. When indicated, control exchange reactions were also performed with purified Lck alone using the same amount of Lck as was used to phosphorylate purified HA-IBP. In some experiments, a water-soluble analogue of PI(3,4,5)P3 (Echelon Research Laboratories Inc.) was added to the exchange reaction mixtures to a final concentration of 1 μM. The exchange reactions were stopped by adding 1 ml of ice-cold dilution buffer (20 mM Tris-HCl, pH 7.5, 100 mM NaCl, 20 mM MgCl2) to the reaction mixtures. The amount of [3H]GDP remaining bound to the GTPases was determined by filtering the quenched reaction samples over nitrocellulose membranes followed by extensive washing of the filters and then quantification of the membrane-bound radioactivity by scintillation counting.

### Example 21

### Conjugate Formation and CD3 Capping

Raji cells were used as the APCs for conjugation with Jurkat T cells. Raji cells were labeled with 10 μM 7-amino-4-chloromethylcoumarin cell tracker blue dye (Molecular Probes, Eugene, Oreg.) followed by pulsing with or without 5 μg/ml SEE (Toxin Technology, Sarasota, Fla.) for 30 min at 37° C. To induce conjugate formation, 1×10⁵ B cells were combined with 1×10⁵ Jurkat E6-1 or 1×10⁵ Lck-deficient Jurkat (J.CaM1.6) T cells at 37° C. for 5 min. Conjugates were pipetted onto poly-L-lysine-coated coverslips and then fixed in 3.7% formaldehyde, washed, and permeabilized with 0.5% Triton X-100/phosphate-buffered saline. Conjugates were then stained with antibodies to IBP and PKC-{theta} followed by a secondary staining with Alexa-Fluor 568-conjugated donkey anti-rabbit (Molecular Probes) and FITC-conjugated donkey anti-mouse (Jackson Immunoresearch Laboratories, IgAb), respectively. For treatments with wortmannin (Calbiochem), T cells were resuspended in serum-free medium containing 100 nM wortmannin and incubated for 30 min at 37° C. followed by incubation with APCs. Conjugates were examined by a Zeiss LSM 510 laser scanning confocal microscope (Thornwood, N.Y.) with a x100/1.3 Plan-Neofluor objective lens. FITC, TRITC, and 7-methyl-4-chloromethylcoumarin (in two-photon mode) were excited at 488, 543, and 800 nm, respectively, and emission was collected at 500-550, above 585, and 435-485 nm, respectively. Optical section thickness was 1 μm. Image enhancement and analysis were performed using the public domain program NIH Image 1.6 and Adobe Photoshop 6.0. Approximately 100 conjugates were scored visually for polarized IBP or PKC-0 at the synapse from two independent scores of three different experiments.

For capping experiments, naïve CD4+ T cells were isolated from splenocytes of D0.11.10 TCR transgenic mice by negative selection using naive CD4+-specific T cell enrichment columns (R & D Systems). The purity of naive CD4+ cells was assessed by flow cytometry and was found to be >90%. T cells were stimulated with 5 μg/ml anti-CD3 {epsilon} Ab (Pharmingen) for 1 h on ice, followed by cross-linking with FITC-labeled mouse anti-hamster Ig Ab (Molecular Probes, Eugene, Oreg.) for 5 min at 4 or 37° C. Cells were pipetted onto poly-L-lysine-coated coverslips and then fixed with 3.7% formaldehyde. Cells were then washed, permeabilized, and stained with antibodies against IBP followed by a secondary antibody stain of anti-rabbit Alexa-Fluor 568 (Molecular Probes). Capped T cells were examined by a Zeiss LSM 510 laser scanning confocal microscope (Thornwood, N.Y.) with a x100/1.3 Plan-Neofluor objective lens. FITC and TRITC were excited at 488 and 543 nm, respectively, and emission was collected at 500-550 and above 585 nm, respectively. Optical section thickness was ˜1 μm. Image enhancement and analysis were performed using the public domain program NIH Image 1.6 and Adobe Photoshop 6.0. The percentage of cells displaying caps, in which the Alexa-Fluor condenses to less than 25% of the cell surface was determined by counting 10 fields per treatment composed of 150-250 cells. D0.11.10 TCR transgenic mice were obtained from Jackson Immunoresearch Laboratories and were maintained under specific pathogen-free conditions.

### Example 22

### IBP is Tyrosine-Phosphorylated Upon TCR Stimulation in an LCK-Dependent Manner

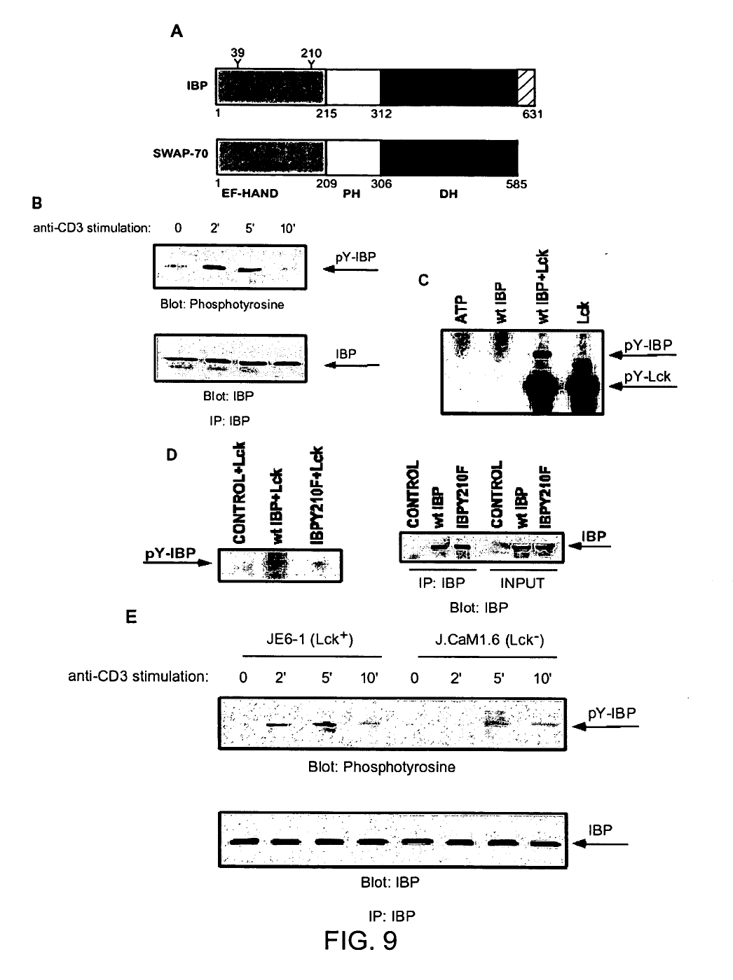

The rapid activation of PTKs of the Src family is one of the earliest signaling events triggered by engagement of the TCR (1, 2). Because a survey of the IBP sequence utilizing the Scansite algorithm (32) revealed the presence of a potential tyrosine phosphorylation site, which fits the consensus motif for Lck-mediated phosphorylation (FIG. 9A) (33), the inventors first determined whether IBP underwent enhanced tyrosine phosphorylation in response to TCR stimulation. Jurkat T cells, which express endogenous IBP, were stimulated with anti-CD3 mAb for varying periods of time, and whole cell lysates were obtained and then immunoprecipitated with an Ab directed against IBP (FIG. 9B). Western blot analysis of the immunoprecipitates with an anti-phosphotyrosine Ab revealed that TCR engagement led to the rapid and transient tyrosine phosphorylation of IBP. To address more directly the possibility that IBP might be a substrate for the Src family of tyrosine kinases, the inventors determined whether purified Lck could phosphorylate purified recombinant IBP in an in vitro kinase assay (FIG. 9C). Coincubation of IBP and Lck resulted in the phosphorylation of IBP in addition to the known autophosphorylation of Lck. Furthermore, transient cotransfection of an IBP expression construct in 293T cells, together with a constitutively active form of Lck (LckY505F) (24), led to the tyrosine phosphorylation of IBP (data not shown). Given that the N terminus of IBP contains a tyrosine (Tyr-210) that represents a potential consensus motif for Lck-mediated phosphorylation, the inventors then proceeded to determine whether inactivating this residue would affect the ability of Lck to phosphorylate IBP. As shown in FIG. 9D, in vitro kinase assays indeed demonstrated that Lck can phosphorylate wild-type IBP but not the Y210F mutant. To assess whether Lck was required for TCR-induced phosphorylation of IBP in lymphocytes, the inventors compared the ability of IBP to undergo tyrosine phosphorylation in cells from the Lck⁺ Jurkat cell line JE6.1 and in cells from J.CaM1.6, an Lck-deficient subline of JE6.1 (34). The anti-CD3-induced tyrosine phosphorylation of IBP was markedly diminished in the Lck-deficient as compared with the Lck⁺ Jurkat T cells (FIG. 9E). Taken together these data indicate that IBP is rapidly tyrosine-phosphorylated upon TCR stimulation and that IBP can serve as a substrate for Src kinases.

### Example 23

### IBP Binds PI(3,4,5)P₃ Upon Phosphorylation by LCK

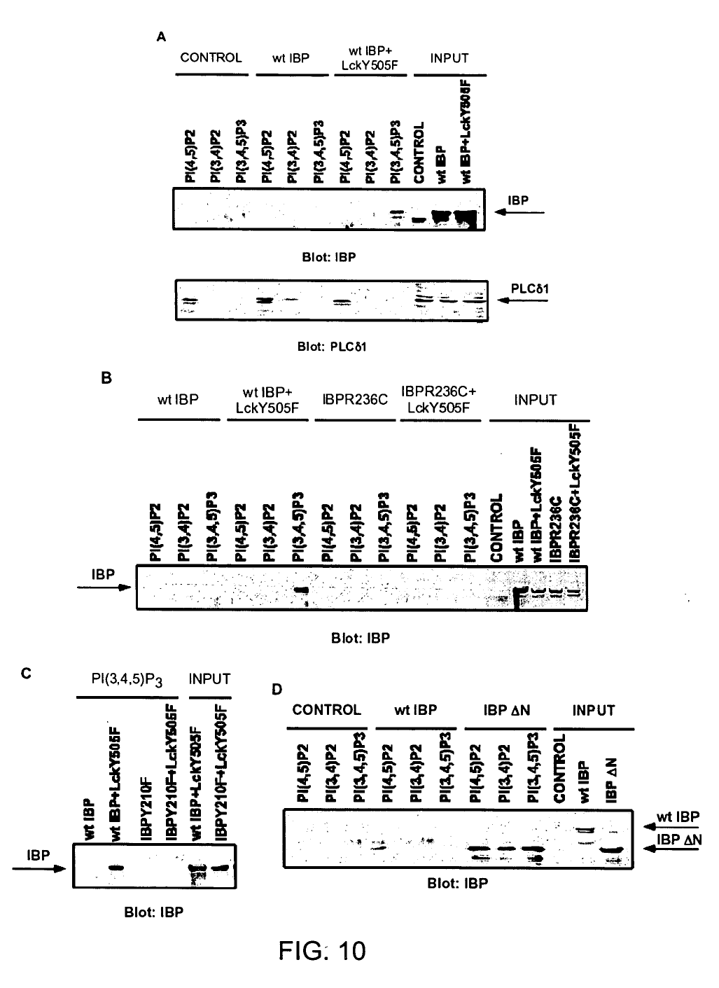

Because IBP contains a PH domain the inventors determined whether IBP binds specific phosphoinositides. The inventors prepared whole cell lysates from 293T cells cotransfected with an HA-tagged IBP expression construct and either an empty vector or a vector expressing a constitutively active Lck (LckY505F). Lysates were then subjected to pull-down assays with a panel of different phosphoinositides conjugated to agarose beads (FIG. 10A). IBP bound efficiently to the PI3K product, PI(3,4,5)P₃, only when coexpressed with constitutively active Lck. The inventors did not detect association of IBP with PI(4,5)P₂. To confirm the specificity of the interaction of PI(3,4,5)P₃ with the PH domain of IBP, the inventors generated a point mutation within the PH domain of IBP (R236C). This residue has been shown previously (35) to be critical for the interaction of PH domains with PI(3,4,5)P₃, and a similar mutation was previously demonstrated to prevent the association of SWAP-70 with phosphoinositides (20). As shown in FIG. 10B, the R236C mutation completely abolished the ability of IBP to interact with PI(3,4,5)P₃. To further investigate whether phosphorylation of Tyr-210 by Lck was critical for the ability of IBP to efficiently associate with PI(3,4,5)P₃ the inventors then assayed the ability of the IBPY210F mutant to bind PI(3,4,5)P₃ (FIG. 10C). Consistent with the notion that Tyr-210 constitutes the major Lck phosphorylation site within the IBP molecule, cotransfection of Lck with IBPY210F did not lead to binding of this mutant to PI(3,4,5)P₃. Interestingly, a deletion mutant of IBP lacking the entire N terminus (IBP ΔN) bound to PI(3,4,5)P₃ even in the absence of constitutively active Lck (FIG. 10D). Surprisingly, this mutant also acquired the ability to bind PI(4,5)P₂ and PI(3,4)P₂. Further studies are in progress to determine whether the binding of IBP ΔN to PI(4,5)P₂ and PI(3,4)P₂ is mediated directly by the PH domain of IBP or by association of this mutant with additional PH domain-containing proteins of different specificities. Collectively, these data are thus consistent with a model whereby phosphorylation of Tyr-210 by Lck leads to a conformational change in IBP that allows its PH domain to bind to the appropriate phosphoinositide molecule.

### Example 24

### Recruitment of IBP to the IS Requires the Activities of LCK and PI3K

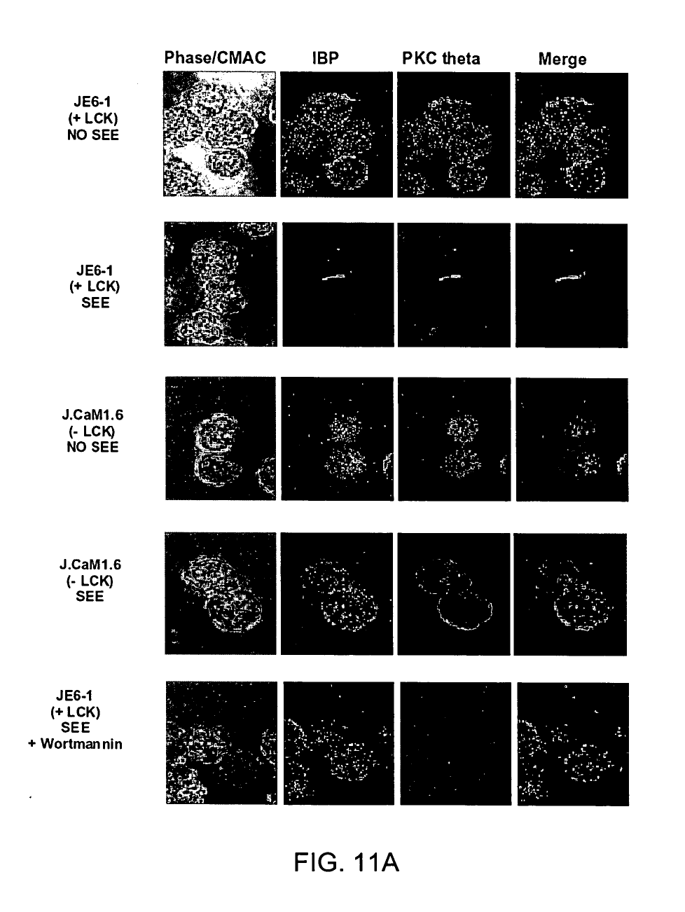

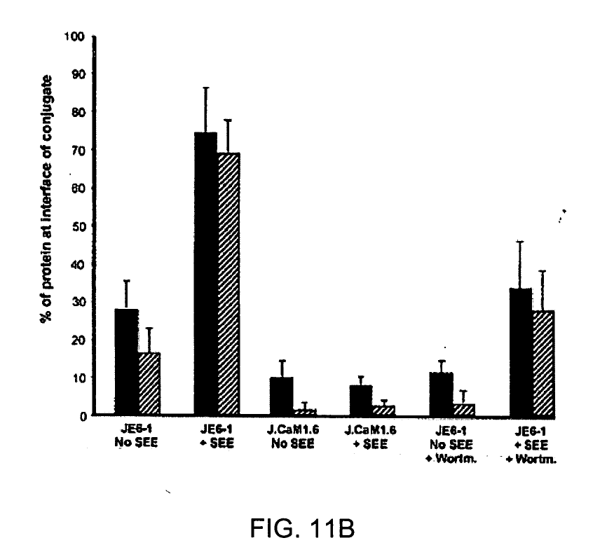

To determine whether IBP is recruited to the immunological synapse, the inventors examined the distribution pattern of endogenous IBP in conjugates formed by Jurkat T cells incubated with APCs (Raji) in the presence or absence of Staphylococcus enterotoxin E (SEE) superantigen (FIG. 11A). The localization of PKC-0, a key molecule known to be recruited to the IS upon T cell activation, was examined, as well (35). In most conjugates formed in the absence of SEE, both IBP and PKC-0 displayed a uniform speckled pattern. However, conjugates formed using SEE-pulsed APCs displayed a striking redistribution of IBP to the synapse. Redistribution of IBP paralleled that of PKC-0, and the two proteins were found to colocalize. Relocalization of IBP and PKC-0 to the IS was observed in ˜75% of activated T cells (FIG. 11B). Interestingly, in 20-30% of conjugates formed in the absence of SEE, the inventors observed some redistribution of both IBP and PKC-θ to the cell:cell contact zone, suggesting that TCR-independent pathways may also contribute to the redistribution of IBP. Consistent with the biochemical studies described above, no recruitment of IBP to the T cell-APC interface could be detected in Lck-deficient mutant Jurkat T cells (J.CaM1.6) upon incubation with SEE-pulsed B cells. Furthermore, addition of a PI3K inhibitor, wortmannin, to Lck⁺ Jurkat T cells (JE6-1) inhibited recruitment of IBP and of PKC-θ to the IS. Taken together, these data indicate that IBP relocalizes to the T cell-APC contact area upon activation and that the activities of both Lck and PI3K are required for the recruitment of IBP to the immunological synapse.

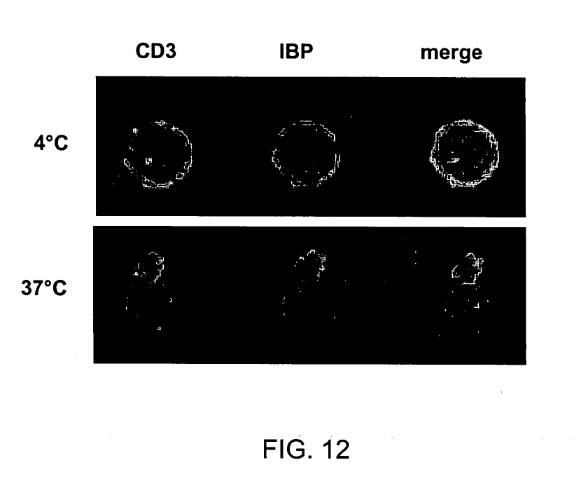

The inventors also examined whether in primary T cells IBP colocalizes with TCR/CD3 caps, asymmetric structures formed by the clustering of T cell receptors in response to T cell activation. Formation of TCR/CD3 caps closely mirrors the molecular mechanisms, which are necessary for synapse formation (37). TCR/CD3 capping was induced in purified CD4⁺ T cells from DO.11.10 TCR Tg mice. In unstimulated cells, both CD3 and IBP molecules were distributed uniformly (FIG. 12 upper panel). Once T cell stimulation was induced, a clear polarization of both TCR/CD3 and IBP to one side of the cell could be observed (FIG. 12, lower panel). IBP colocalized with the TCR/CD3 cap in ˜70% of the cells (data not shown). These results indicate that activation of primary T cells leads to the relocalization of IBP to areas of TCR clustering where critical downstream signaling events occur.

### Example 25

### BP Exhibits GEF Activity Toward RAC1 and CDC42 but not Toward RHOA

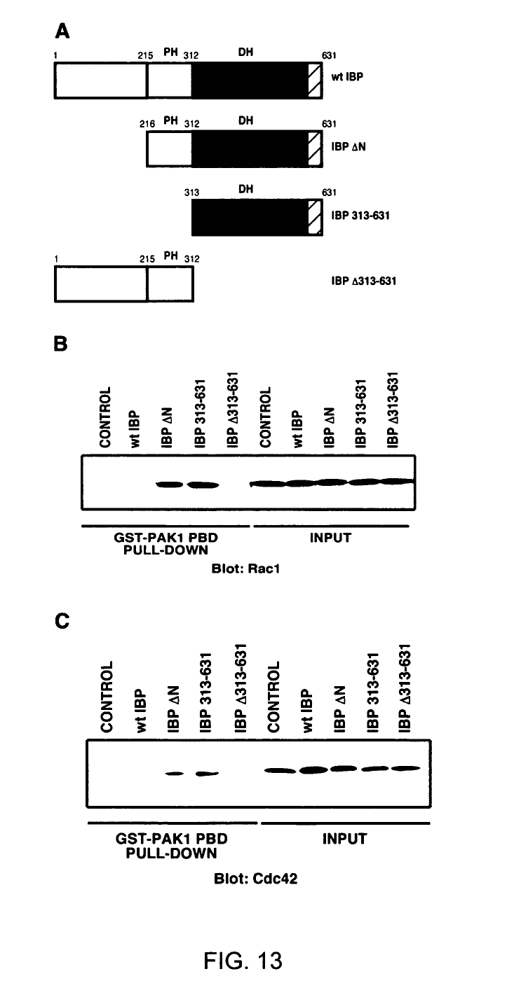

Activation of Rho GTPases plays a critical role in the complex cytoskeletal dynamics that underlie the formation, as well as the function, of the IS (49, 50). Because IBP shares a high degree of similarity with SWAP-70, a protein known to possess GEF activity toward Rac1 (27), the inventors determined whether IBP activates specific members of the Rho family of proteins. The inventors generated a panel of IBP deletion mutants that removed either the potentially autoinhibitory N-terminal region (IBP ΔN) or the putative C-terminal DH domain (IBP Δ313-631) (FIG. 13A). The inventors also generated a mutant (IBP 313-631) containing the putative DH domain alone. The inventors utilized these constructs in in vivo assays of GEF activity (FIG. 13, B and C). Wild-type IBP, in the absence of any additional signals, was unable to mediate the activation of Rac1 or Cdc42. However, expression of the deletion mutants that either lacked the potential autoinhibitory N-terminal domain (IBP ΔN) or contained the potential DH domain alone (IBP 313-361) led to Rac1 activation (FIG. 13B) and, more modestly, Cdc42 activation (FIG. 13C). Activation of these GTPases was not detected in COS-7 cells expressing a deletion mutant of IBP lacking the potential DH domain (IBP A313-631). These results indicate that IBP displays GEF activity for Rac1 and Cdc42 in vivo. The GEF catalytic activity of IBP maps to its DH domain, and the N terminus of IBP may contain a regulatory domain that modulates its GEF activity.

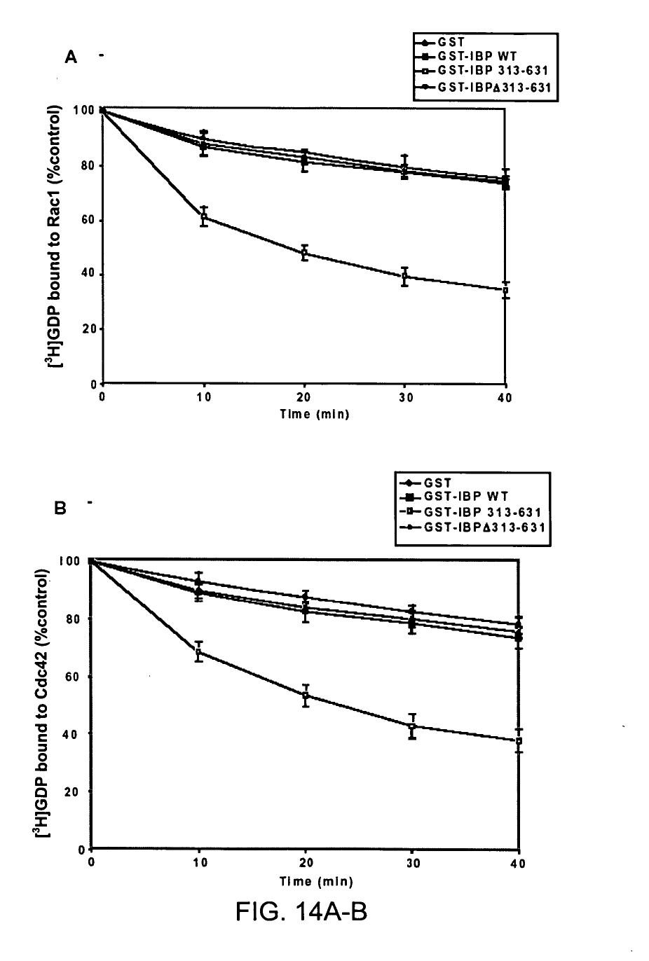

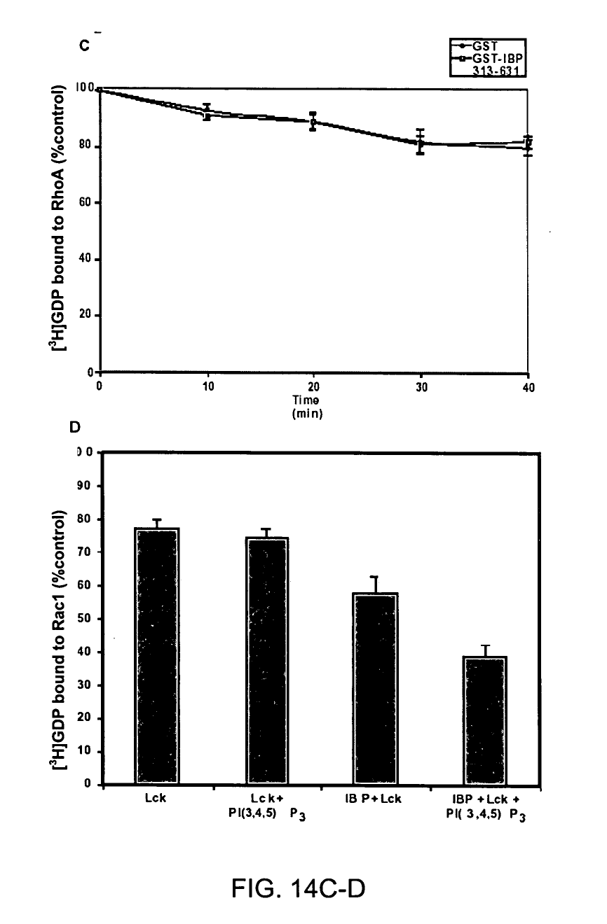

To confirm that IBP can act as a GEF for Rho GTPases, the inventors subsequently utilized purified recombinant wild-type or mutant IBP proteins to conduct in vitro GDP release assays on purified recombinant Rho proteins. Consistent with the in vivo activation assays, incubation of wild-type IBP with Rac1 (FIG. 14A) or Cdc42 (FIG. 14B) did not stimulate the dissociation of [³H]GDP from these Rho GTPases. However, a mutant form of IBP containing only the putative DH domain (313-631) stimulated rapid GDP dissociation from both Rac1 (FIG. 14A) and Cdc42 (FIG. 14B) but not from RhoA (FIG. 14C). In contrast, an IBP deletion mutant lacking this C-terminal domain (IBP Δ313-631) did not exhibit any GDP/GTP exchange activity on Rac1 or Cdc42, further supporting the notion that the GEF activity of IBP is contained within its C terminus. Because the inventors' initial studies had demonstrated that both Lck and PI3K enzymes target IBP, the inventors examined the potential impact of these signaling pathways on the GEF activity of IBP (FIG. 14D). Although in the absence of any additional signal, the wild-type IBP molecule did not display any GEF activity, phosphorylation-mediated activation of IBP by Lck resulted in an enhanced ability of the wild-type IBP to promote GDP-GTP exchange on Rac1 (FIG. 14D). Addition of the PI3K product PI(3,4,5)P₃ in the exchange reactions stimulated the GEF activity of Lck-phosphorylated wild-type IBP on Rac 1 even further (FIG. 14D). These results indicate that IBP can possess GEF activity toward Rac1 and Cdc42 but not toward RhoA. Furthermore, the signals that control the recruitment of IBP to the immunological synapse also regulate its GEF activity.

Discussed below are results obtained by the inventors in connection with the experiments of Examples 1-21:

It has become clear that the productive interaction of a CD4⁺ T cell with an antigen-presenting cell results in the profound reorganization of receptors and signaling molecules leading to the formation of what has been termed the immunological synapse (9, 10, 11). Remodeling of the actin cytoskeleton by the Rho family of GTPases is fundamental to the achievement of this three-dimensional structure. Because Rho GTPases are major regulators of the actin cytoskeleton, precise regulation of their activity is likely to be essential to the assembly/function of the immunological synapse. Here the inventors report that IBP is a novel type of GEF that is activated in response to TCR engagement and is recruited to the immunological synapse.

The ability of IBP to activate Rac1 and Cdc42 is consistent with the fact that IBP displays a high degree of similarity to SWAP-70, a protein recently described to represent a novel type of GEF for Rac1 (27). SWAP-70 and IBP may be the only two members of this unique class of GEFs, because the inventors have failed to identify any additional sequences sharing significant homology with these two proteins despite extensive searches. Interestingly, during the course of these studies Tanaka et al. (69) reported the identification of a protein homologous to SWAP-70, which they termed SLAT (SWAP-70like adapter of T cells). A comparison of the sequences of these two proteins has revealed that murine IBP is identical to SLAT. Strikingly, expression studies have indicated that, unlike SWAP-70, IBP is expressed in T cells suggesting that IBP may be the predominant member of this unique class of GEFs to be found in T lymphocytes (26, 29, 69). Furthermore, although both SWAP-70 and IBP can be detected in B cells, these two molecules appear to be expressed at distinct stages of B cell differentiation as indicated by the fact that IBP is largely absent in germinal center B cells, which strongly express SWAP-70 (26, 29). The differential usage of these two GEFs by the immune system in specific compartments suggests that the functional outcome of the signaling events elicited by these two GEFs may be distinct. An alternative explanation is that the regulatory requirements for the activation of IBP and SWAP-70 are different. Indeed, whereas the GEF activity of IBP is controlled by both tyrosine phosphorylation and phosphoinositide binding (FIG. 14D), activation of SWAP-70 has been reported to depend primarily on the activity of PI3K (27). The generation of IBP-deficient mice should provide insight into whether these two GEFs play unique or redundant roles in the immune system.

The regulation of IBP in response to TCR signaling is reminiscent of the regulation of Vav, whose activity is controlled by both tyrosine phosphorylation and phosphoinositide binding (60, 61). As in the case of Vav, whose phosphorylation by either Src or Syk tyrosine kinases leads to the relief of an intramolecular inhibitory interaction (23), these results indicate that tyrosine phosphorylation of IBP by Lck is also likely to lead to a conformational change. This notion is supported by the finding that deletion of the N terminus leads to a constitutively active form of IBP whereas mutation of Tyr-210, the major Lck-phosphorylation site, prevented its ability to interact with PI(3,4,5)P₃. Although both Vav and IBP contain a PH domain and bind the PI3K product PI(3,4,5)P₃, these studies suggest that the exact mechanism by which phosphoinositides participate in the activation of these two GEFs may differ. Although binding of PI(3,4,5)P₃ is believed to enhance the tyrosine phosphorylation of Vav by PTKs (61), tyrosine phosphorylation appears to be a prerequisite for the ability of IBP to bind phosphoinositides. Therefore, the inventors favor a model whereby the N-terminal region of IBP serves a regulatory function and maintains IBP in a closed conformation until its TCR-induced tyrosine phosphorylation relieves this autoinhibitory interaction. This would then expose the PH domain allowing it to bind PI(3,4,5)P₃. Binding of this phosphoinositide would lead to full activation of IBP by enabling its DH domain to interact with the appropriate Rho GTPase, as well as by directing IBP to the appropriate subcellular compartment.

The fact that IBP and Vav respond to TCR-mediated signals, and both activate Rac1 and Cdc42, raises the critical question of the exact role that IBP plays in T cells. Although genetic studies will be required to definitely address this issue, it has been suggested that GEFs may not only activate the GTPases but also regulate the downstream pathways that these GTPases control (24). This may be because of the fact that specific GEF/GTPase combinations, may elicit only a limited subset of the large number of downstream effector pathways activated by these GTPases. Given the complexities that underlie the assembly, as well as the functions of the immunological synapse, recruitment and activation of distinct GEFs would thus enable T cells to precisely control the molecular processes mediated by the synapse.

Although the inventors' initial studies have focused on two specific sets of signals, those mediated by Lck and PI3K, IBP function, like that of Vav (23), may be regulated by additional mechanisms and/or stimuli, as well. Vav can be tyrosine-phosphorylated not only by Lck but also by another TCR-activated PTK, ZAP-70 (70). It is thus possible that additional kinases may also phosphorylate IBP. The presence of a putative EF-hand motif at the N terminus furthermore suggests that calcium may also play a role in the regulation of IBP function. Finally, the inventors have noted in the inventors' conjugate experiments that even in the absence of SEE, redistribution of IBP to the contact zone was observed in 20-30% of cells. This is reminiscent of proteins such as ezrin whose localization is controlled not just by TCR signals but also by integrin engagement (71). Because cross-talk between TCR and additional coreceptors such as the integrin LFA-1 is crucial for synapse formation and T cell activation, determining the spectrum of receptors that activate IBP will be essential to understand the role of this novel GEF in T cell function.

While the foregoing invention has been described in some detail for purposes of clarity and understanding, it will be appreciated by one skilled in the art, from a reading of the disclosure, that various changes in form and detail can be made without departing from the true scope of the invention in the appended claims.

The following references are cited and incorporated by reference herein in their entirety:
1. van Leeuwen. J., and L. E. Samelson. 1999. T cell antigen-receptor signal transduction. Curr. Opin. Immunol. 11:242-248.
2. Kane. L. P., J. Lin, and A. Weiss. 2000. Signal transduction by the TCR for antigen. Curr. Opin. Immunol. 12:242-249.
3. Leitenberg, D., and K. Bottomly. 1999. Regulation of naive T cell differentiation by varying the potency of TCR signal transduction. Sem. Immunol. 11:283-292.
4. Szabo, S.3., B. M. Sullivan, S. L. Peng, and L. H. Glimcher. 2003. Molecular mechanisms regulating Th1 immune responses. Annu. Rev. Immunol. 21:713-758.
5. Murphy, K. M., and S. L. Reiner. 2002. The lineage decisions of helper T cells. Nat Rev Immunol. 2:933-944.
6. Fuller, C. L., V. L. Braciale, and L. E. Samelson. 2003. AU roads lead to actin: the intimate relationship between TCR signaling and the cytoskeleton. Immunol Rev. 191:220-236.
7. Cannon, J. L., and l. K. Burkhardt. 2002. The regulation of actin remodeling during T-cell APC conjugate formation. Immunol Rev. 186:90-99.
8. Krawczyk, C., and J. M. Penttinger. 2001. Molecular motors involved in T cell receptor clusterings. J. Leukoc. Biol. 69:317-330.
9. Bromley, S. K., W. R. Burack, K. G. Johnson, K. Somersalo, T. N. Sims, C. Sumen, M. M. Davis, A. S. Shaw, P. M. Allen, and M. L. Dustin. 2001. The immunologic synapse. Annu. Rev. Immunol. 19:375-396.
10. van der Mei-we, P. A. 2002. Formation and function of the immunological synapse. Curr. Opin. Immunol. 14:293-298.
11. Delon, J., and R. N. Germain. 2000. Information transfer at the immunological synapse. Curr. Biol. 10:R923-R933.
12. Smyth, L. A., L. Ardouin, O. Williams, T. Norton, V. Tybulewiez, and D. Kioussis. 2002. Inefficient clustering of tyrosine-phosphorylated proteins at the immunological synapse in response to an antagonist peptide. Eur. J. inznzunol. 32:3386-3394.
13. Ehrlich, L. L R., P. J. R. Ebert, M. F. Krummel, A. Weiss, and M. M. Davis. 2002. Dynamics of p561ck translocation to the T cell immunological synapse following agonist and antagonist stimulation. Immunity 17:809-822.
14. Symons, M., and J. Settleman. 2000. Rho family GTPases: more than simple switches. Trends Cell Biol. 10:415-419.
15. Ridley, A. J. 2001. Rho family proteins: coordinating cell responses. Trends Cell Biol. 11:471-477.
16. Takai, Y., T. Sasaki, and T. Matozaki. 2001. Small GTP-binding proteins. Physiol. Rev. 81:153-208.
17. Bustelo, X. R. 2002. Understanding Rho/Rae biology in T-cells using animal models. Bioessays 24:602-612.
18. Cantrell, D. R. 2003. GTPases and T cell activation. Immunol Rev. 192:122-130.
19. Aznar, S., and J. C. Lacal. 2001. Rho signals to cell growth and apoptosis. Cancer Letters 1651-10.
20. Zheng, Y. 2001. Dbl family guanine nucleotide exchange factors. TIES 26:724-732.
21. Fischer, K.-D., K. Tedford, and J. M. Penninger. 1998. Vav links antigen-receptor signaling to the actin cytoskeleton. Son. Immunol. 10:317-327.
22. Bustelo, X. R. 2001, Vav proteins, adaptors and cell signaling. Oncogene 20:6372-6381.
23. Turner, M., and D. D. Billadeau. 2002. Vav proteins as signal integrators for multi-subunit immune-recognition receptors. Nature Rev. Immunol. 2:476-486.
24. Schmidt, A., and A. Hall. 2002. Guanine nucleotide exchange factors for. Rho GTPases: turning on the switch. Genes Dev. 16:1587-1609.

25. Reif, K., and D. A. Cantrell. 1998. Networking Rho family GTPases in lymphocytes. Immunity 8:395-401.
26. Gupta, S., A. Lee, C. flu, J. Fanzo, I. Goldberg, G. Cattoretti, and A. B. Pernis. 2003. Molecular cloning of IBP, a SWAP-70 homologous GEF, which is highly expressed in the immune system. Human bnmunol. 64:389-401.
27. Shinohara, M., Y. Terada, A. Iwamatsu, A. Shihora, N. Mochizuki, M. Higuchi, Y. Gotoh, S. Ihara, S. Nagata, H. Itoh, Y. Fukui, and R. Jessberger. 2002. SWAP-70 is a guanine-nucleotide-exchange factor that mediates signalling of membrane ruffling. Nature 416:759-763.
28. Borggrefe, T., S. Keshavarzi, B. Gross, M. Wabl, and R. Jessberger. 2001. Impaired IgE response in SWAP-70-deficient mice. Eur. J. Immunol. 31:2467-2475.
29. Borggrefe. T., L. Masat, M. Wabl, B. Riwar, G. Cattoretti, and R. Jessberger. 1999. Cellular, intracellular, and developmental expression patterns of murine SWAP-70. Eur. J. Immunol. 29:1812-1822.
30. Gupta, S., J. C. Fanzo, C. flu, D. Cox, S. Y. Jang, A. B. Lee, S. Greenberg, and A. B. Pemis. 2003. T cell receptor engagement leads to the recruitment of IBP, a novel guanine nucleotide exchange factor, to the immunological synapse. J. Biol. Chem. 278:43541-43549.
31. Tanaka, Y., K. Bi, R. Kitamura, S. Hong, Y. Altman, A. Matsumoto, H. Tabata, S. Lebedeva, P. J. Bushway, and A. Altman. 2001 SWAP-70-like adapter of T cells, an adapter protein that regulates early TCR-initiated signaling in Th2 lineage cells. immunity 18:403-414.
32. Zambrowicz, B Y., G. A. Friedrich, E. C. Buxton, S. L. Lilleberg, C. Person, and A. T. Sands. 1998. Disruption and sequence identification of 2,000 genes in mouse embryonic stem cells. Nature 392:608-611.
33. l-Ioldorf, A. D., K.-H. Lee, R. Bttrack, P. M. Allen, and A. S. Shaw. 2002. Regulation of Lck activity by CD4 and CD28 in the immunological synapse. Nature Immunol. 3:259-264.

34. Costa, G. L., J. M. Benson, C. M. Seroogy, P. Achacoso, C. G. Fathman, and G. P. Nolan. 2000. Targeting rare populations of murine antigen-specific T lymphocytes by retroviral transduction for potential application in gene therapy for autoimmune disease. J. Immunol. 164:3581-3590.
35. Su, B., and M. Karin. 1996. Mitogen-activated protein kinase cascades and regulation of gene expression. Curr. Opin. Immunol. 8:402-411.
36. Li, B., H. Yu, W. Zheng, R. Vol], S. Na, A. W. Roberts, D. A. Williams, R. J. Davis, S. Gh.osh, and R. A. Flavell. 2000. Role of the guanosine triphosphatase Rac2 in T helper 1 cell differentiation. Science 288:2219-2222.
37. Jorritsma, P, J., J. L. Brogdon, and K. Bottomly. 2003. Role of TCR-induced extracellular signa-regulated kinase activation in the regulation of early IL-4 expression in naive CD4t T cells. J. Immunol. 170:2427-2434.
38. Fenix, L. A., M. T. Sweetser, W. M. Weaver, J. P. Hoeffler, T. K. Kerppola, and C. B. Wilson. 1996. The proximal regulatory element of the interferon-y promoter mediates selective expression in T cells. J. Biol. Chem. 271:31964-31972.
39. Huppa, J. B., M. Gleimer, C. Sumen, and M. M. Davis. 2003. Continous T cell receptor signaling required for synapse maintenance and full effector potential. Nature Immunol. Tskvitaria, l., A. L. Rozelle, H. L. Yin, and C. Wulfing. 2003. Regulation of sustained actin dynamics by the TCR and costimulation as a mechanism of receptor localization. J. Immunol. 171:2287-2295.
40. Fischer, K. D., Y. Y. Kong, H. Nishina, K. Tedford, L. E. Marengere, I. Kozieradzki, T. Sasaki, M. Starr, G. Chan, S. Gardener, M. P. Nghiem, D. Bouchard, M. Barbacid, A. Bernstein, and J. M. Penninger. 1998. Vav is a regulator of cytoskeletal reorganization mediated by the T-cell receptor. Curr Biel 8:554-562.
41. Holsinger. L. J., L A. Graer, W. Swat, T. Chi, D. M. Bautista, L. Davidson, R. S. Lewis, F. W. Alt, and G. R. Crabtree. 1998. Defects in actin-cap formation in Vav-defient mice implicate an actin requirement for lymphocyte signal transduction. Curr. Biol. 8:563-572.
42. Wulfing, C., A. Bauch, G. R. Crabtree, and M. M. Davis. 2000. The vav exchange factor is an essential regulator in actin-dependent receptor translocation to the lymphocyteantigen-presenting cell interface. Proc Nall Acad Sci USA. 97:10150-10155.
43. Costello, P. S., A. E. Walters, Y. J. Mee, M. Turner, L. F. Reynolds, A. Frisco, N. Sarner, R. Zamoyska, and V. L. Tybulewicz. 1999. The Rho-family GTP exchange factor Vav is a critical transducer of T cell receptor signals to the calcium, ERK, and NF-kappaB pathways. Proc Mal Acad Sri USA 96:3035-3040.
44. Fujikawa, K., A. V. Miletic, P. W. Alt, R. Faccio, T. Brown, J. Hoog, J. Fredericks, S. Nishi, S. Mildiner, S. L. Moores, J. Brugge, F. S. Rosen, and W. Swat. 2003. Vav1/2/3-null mice define an essential role for Vav family proteins in lymphocyte development and activation but a differential requirement in MAPK signaling in T and B cells. J. Exp. Med. 198:1595-1608.
45. Kuhne, M. R., G. Ku, and A. Weiss. 2000. A guanine nucleotide exchange factor-independent function of Vnv1 in transcriptional activation. 0.1 Biol. Chem. 275:2185-2190.
46. Clements, J. L., and Koretzky, G. A. (1999) J. Clin. Invest. 103:925-929.
47. Cantley, L. C. (2002) Science 296, 1655-1657.
48. Ward, S. G., and Cantrell, D. A. (2001) Curr. Opin. Immunol. 13:332-338.
49. Dustin, M. L., and Cooper, J. A. (2000) Nat. Immunol. 1:23-29.
50. Krawczyk, C., and Penninger, J. M. (2001) J. Leukocyte Biol. 69:317-330.
51. Zheng, Y. (2001) Trends Biochem. Sci. 26:724-732.
52. Hoffman, G. R., and Cerione, R. A. (2002) FEBS Lett. 513:85-91.
53. Hakeda-Suzuki, S., Ng, J., Tzu, L., Dietzl, G., Sun, Y., Harms, M., Nardine, T., Luo, L., and Dickson, B. J. (2002) Nature 416:438-442.
54. Liu, Y., Witte, S., Liu, Y. C., Doyle, M., Elly, C., and Altman, A. (2000) J. Biol. Chem. 275:3603-3609.
55. Abr:am, K., Levin, S., Marth, J., Forbush, K., and Perlmutter, R. (1991) Proc. Natl. Acad. Sci. U.S.A. 88:3977-3981.
56. Gupta, S., Jiang, M., Anthony, A., and Pemis, A. (1999) J. Exp. Med. 190:1837-1848.
57. Gupta, S., Xia, D., Jiang, M., Lee, S., and Pernis, A. (1998) J. Immunol. 161:5997-6004.
58. Qian, D., Lev, S., van Oers, N. S., Dikic, I., Schlessinger, J., and Weiss, A. (1997) J. Exp. Med. 185:1253-1259.
59. Shou, C., Farnsworth, C. L., Neel, B. G., and Feig, L. A. (1992) Nature 358:351-354.
60. Crespo, P., Schuebel, K. E., Ostrom, A. A., Gutkind, J. S., and Bustelo, X. R. (1997) Nature 385:169-172.
61. Han, J., Luby-Phelps, K., Das, B., Shu, X., Xia, Y., Mosteller, R. D., Krishna, U. M., Falck, J. R., White, M. A., and Broek, D. (1998) Science 279:558-560.
62. Abe, K., Rossman, K. L., Liu, B., Ritola, K. D., Chiang, D., Campbell, S. L., Burridge, K., and Der, C. J. (2000) J. Biol. Chem. 275:10141-10149.
63. Yaffe, M. B., Leparc, G. G., Lai, J., Obata, T., Volinia, S., and Cantley, L. C. (2001) Nat. Biotechnol. 19:348-353.
64. Latour, S., and Veillette, A. (2001) Curr. Opin. Immunol. 13:299-306.
65. Straus, D. B., and Weiss, A. (1992) Cell 70:585-593.
66. Isakoff, S. J., Cardozo, T., Andreev, J., Li, Z., Ferguson, K. M., Abagyan, R., Lemmon, M. A., Aronheim, A., and Skolnik, E. Y. (1998) EMBO J. 17:5374-5387.
67. Monks, C. R., Kupfer, H., Tamir, I., Barlow, A., and Kupfer, A. (1997) Nature 385:83-86.
68. Bauch, A., Alt, F. W., Crabtree, G. R., and Snapper, S. B. (2000) Adv. Immunol. 75:89-114.
69. Tanaka, Y., Bi, K., Kitamura, R., Hong, S., Altman, Y., Matsumoto, A., Tabata, H., Lebedeva, S., Bushway, P. J., and Altman, A. (2003) Immunity 18:403-414.
70. Deckert, M., Tartare-Deckert, S., Couture, C., Mustelin, T., and Altman, A. (1996) Immunity 5:591-604.
71. Roumier, A., Olivo-Marin, J. C., Arpin, M., Michel, F., Martin, M., Mangeat, P., Acuto, O., Dautry-Varsat, A., and Alcover, A. (2001) Immunity 15:715-728.

What is claimed is:

1. A method of modulating T cell receptor (“TCR”) dependant regulation of an effecting factor in a T cell, comprising administering to the T cell an IBP modulator in an amount effective to modulate the function of IBP, wherein the effecting factor is selected from the group consisting of CD25, CD69, Cdc42, ERK1, ERK2, actin, c-Fos, IFN-γ, IgE, IgG, IL-2, LAT, Rac1, and ZAP-70.

2. The method of claim 1, wherein the IBP modulator is an IBP inhibiting agent, and wherein the administering of the IBP inhibiting agent reduces TCR dependant up-regulation of an effecting factor, wherein the effecting factor is selected from the group consisting of CD25, CD69, Cdc42, ERK1, ERK2, F-actin, c-Fos, IFN-γ, IL-2, LAT, Rac1, and ZAP-70.

3. The method of claim 1, wherein the IBP modulator is an IBP augmenting agent, and wherein the administering of the IBP augmenting agent increases TCR dependant up-regulation of an effecting factor, wherein the effecting factor is selected from the group consisting of CD25, CD69, Cdc42, ERK1, ERK2, F-actin, c-Fos, IFN-γ, IL-2, LAT, Rac1, and ZAP-70.

4. The method of claim 1, wherein the IBP modulator is an IBP inhibiting agent, and wherein the administering of the IBP inhibiting agent increases TCR dependant up-regulation of an effecting factor, wherein the effecting factor is selected from the group consisting of IgE and IgG.

5. The method of claim 1, wherein the IBP modulator is an IBP augmenting agent, and wherein the administering of the IBP augmenting agent reduces TCR dependant up-regulation of an effecting factor, wherein the effecting factor is selected from the group consisting of IgE and IgG.

6. The method of claim 1, wherein the IBP modulator is selected from the group consisting of an expression vector comprising a nucleic acid encoding an IBP modulating agent, a gene knockout vector, a gene expression silencing agent, a gene expression enhancing agent, an IBP inhibitor, and an IBP augmenter.

7. The method of claim 6, wherein the IBP modulating agent is selected from the group consisting of an antisense nucleic acid, an interference RNA, an antibody, a gain-of-function IBP mutant, and a loss-of-function IBP mutant.

8. The method of claim 6, wherein the gene expression silencing agent is at least one of an antisense nucleic acid and an interference RNA.

9. The method of claim 6, wherein the IBP inhibitor is selected from the group consisting of a small molecule IBP inhibitor, a protein phosphatase, a IBP antibody, and a loss-of-function IBP mutant.

10. The method of claim 6, wherein the IBP augmenter is selected from the group consisting of a small molecule IBP augmenter, a protein kinase, and a gain-of-function IBP mutant.

11. The method of claim 10, wherein the IBP augmenter is Lck.

12. The method of claim 11, wherein the function of the IBP is further enhanced by binding phosphatidylinositol 3,4,5-triphosphate (“PI(3,4,5)P₃”).

13. A method of modulating the proliferation and/or differentiation of a T cell comprising administering to the T cell an IBP modulator in an amount effective to modulate the function of IBP.

14. The method of claim 13, wherein the proliferation and/or differentiation of a T cell is TCR dependent.

15. The method of claim 14, wherein the TCR dependent T cell differentiation is a TH 1 differentiation, and wherein the TH 1 differentiation is inhibited by down-regulating IBP in the T cell using the IBP modulator.

16. The method of claim 14, wherein the TCR dependent T cell differentiation is a TH2 differentiation, and wherein the TH2 differentiation is enhanced by up-regulating IBP in the T cell using the IBP modulator.

17. A method for identifying a modulator of IBP-Lck interaction, comprising:
(a) administering a candidate agent and IBP to an in vitro system comprising Lck; and
(b) determining the effect of the candidate agent on Lck catalyzed IBP phosphorylation.

18. A kit for use in identifying a modulator of IBP-Lck interaction, comprising:
(a) IBP;
(b) Lck;
(c) at least one kinase assay reagent; and
(d) instructions for using the kit.

19. A method for identifying a modulator of IBP-PI(3,4,5)P₃ interaction, comprising:
(a) administering a candidate agent and IBP to an in vitro system comprising PI(3,4,5)P₃; and
(b) determining the effect of the candidate agent on IBP-PI(3,4,5)P₃ interaction.

20. A kit for use in identifying a modulator of IBP-PI(3,4,5)P₃ interaction, comprising:
(a) IBP;
(b) PI(3,4,5)P₃; and
(c) instructions for using the kit.

21. A method for identifying a modulator of an effecting factor in a T cell, comprising:
(a) administering a candidate agent to the T cell comprising the effecting factor, wherein the candidate agent is selected from the group consisting of an expression vector comprising a nucleic acid encoding an candidate IBP modulating agent, a candidate IBP gene expression silencing agent, a candidate IBP gene expression enhancing agent, a candidate IBP inhibitor, and a candidate IBP augmenter; and
(b) determining the effect of the candidate agent on the effecting factor, wherein the effecting factor is selected from the group consisting of CD25, CD69, Cdc42, ERK1, ERK2, F-actin, c-Fos, IFN-γ, IgE, IgG, IL-2, LAT, Rac1, and ZAP-70.

22. A composition comprising the modulator of claim 21.

23. The composition of claim 22, further comprising a pharmaceutically-acceptable carrier.

24. A composition comprising an IBP modulator, wherein the IBP modulator modulates TCR dependant regulation of at least of an effecting factor in a T cell, and wherein the effecting factor is selected from the group consisting of CD25, CD69, Cdc42, ERK1, ERK2, F-actin, c-Fos, IFN-γ, IgE, IgG, IL-2, LAT, Rac1, and ZAP-70.

25. The composition of claim 24, further comprising a pharmaceutically-acceptable carrier.

26. A composition comprising an IBP modulator, wherein the IBP modulator modulates the proliferation and/or differentiation of a T cell.

27. The composition of claim 26, further comprising a pharmaceutically-acceptable carrier.
